(19) United States
(12) Patent Application Publication
(10) Pub. No.: US 2017/0088882 A1
(43) Pub. Date: Mar. 30, 2017

(54) CARRIER FOR DETECTING FOODBORNE-ILLNESS-CAUSING BACTERIA, KIT FOR DETECTING FOODBORNE-ILLNESS-CAUSING BACTERIA, METHOD FOR DETECTING FOODBORNE-ILLNESS-CAUSING BACTERIA, AND PCR REACTION SOLUTION FOR FOODBORNE-ILLNESS-CAUSING BACTERIA

(71) Applicant: TOYO SEIKAN GROUP HOLDINGS, LTD., TOKYO (JP)

(72) Inventors: Kazuki Nakajima, Kanagawa (JP); Takaaki Yamasaki, Kanagawa (JP); Satoshi Furukawa, Kanagawa (JP)

(73) Assignee: TOYO SEIKAN GROUP HOLDINGS, LTD., TOKYO (JP)

(21) Appl. No.: 15/375,779

(22) Filed: Dec. 12, 2016

Related U.S. Application Data
(63) Continuation of application No. PCT/JP2015/002931, filed on Jun. 11, 2015.

(30) Foreign Application Priority Data
Jun. 11, 2014 (JP) 2014-120918
Jun. 11, 2014 (JP) 2014-120919

(51) Int. Cl. C12Q 1/68

(52) U.S. Cl. CPC C12Q 1/689; C12Q 2600/158; C12Q 2600/16

(57) ABSTRACT

A carrier for detecting food poisoning bacteria that is used to simultaneously detect Escherichia coli, Salmonella, Staphylococcus aureus, and Vibrio parahaemolyticus includes a plurality of probes comprising: a probe from a region of pyrH gene of Escherichia coli; a probe from a region of vtx1 gene of Escherichia coli; a probe from a region of vtx2 gene of Escherichia coli; a probe from a region of invA gene of Salmonella; a probe from a region of dnaJ gene of Staphylococcus aureus; a probe from a region of toxR gene of Vibrio parahaemolyticus; a probe from a region of tdh gene of Vibrio parahaemolyticus; a probe from a region of trh1 gene of Vibrio parahaemolyticus; and a probe from a region of trh2 gene of Vibrio parahaemolyticus, wherein the probes are immobilized on the carrier.

### TECHNICAL FIELD

The invention relates to technology for detecting microorganisms such as food poisoning bacteria. In particular, the invention relates to a carrier for detecting food poisoning bacteria and a kit for detecting food poisoning bacteria that simultaneously detects Escherichia coli (E. coli), Salmonella, Staphylococcus aureus, and Vibrio parahaemolyticus.

### BACKGROUND ART

In recent years, there has been a downward trend in the incidence of food poisoning along with an improvement in hygiene level in the field of public health (e.g., food and environment). In Japan, however, more than 20,000 people suffer food poisoning every year. A considerable number of food poisoning cases are caused by Escherichia coli, Salmonella, Staphylococcus aureus, and Vibrio parahaemolyticus. Therefore, it is important to accurately and promptly detect the presence or absence of these food poisoning bacteria in food and the environment in order to prevent the occurrence of food poisoning by inspection of food poisoning bacteria.

These food poisoning bacteria may be detected using a method that amplifies the DNA fragment of the amplification target region (amplification target gene region or target gene region) by means of a polymerase chain reaction (PCR) using specific primers, and analyzes the size of the amplified product by means of electrophoresis (see Patent Document 1, for example).

Patent Document 2 (that was applied for by the applicant of the present application) discloses a method that simultaneously amplifies the DNA fragments of eight types of amplification target regions of seven types of food poisoning bacteria including Escherichia coli, Salmonella, Staphylococcus aureus, and Vibrio parahaemolyticus by means of multiplex PCR using specific primers, and analyzes the size of the amplified product by means of electrophoresis.

### RELATED ART DOCUMENTS

### Patent Documents

Patent Document 1: JP-T-2008-538075

Patent Document 2: WO2011/129091

### SUMMARY OF THE INVENTION

In the field of public health, it is desirable to determine the presence or absence of pathogenic Escherichia coli (e.g., enterohemorrhagic Escherichia coli) and pathogenic Vibrio parahaemolyticus that may cause food poisoning, and also determine the presence or absence of these bacteria at the same time as a hygienic index irrespective of pathogenicity. However, it is impossible to detect Escherichia coli and Vibrio parahaemolyticus that may be non-pathogenic together with enterohemorrhagic Escherichia coli and pathogenic Vibrio parahaemolyticus using known technology.

Since various types of DNA (e.g., food genes) are mixed in a sample at a site in which the presence or absence of food poisoning bacteria is determined, detection accuracy with higher specificity has been desired.

In known detection methods, the detection accuracy of Staphylococcus aureus may be decreased. Specifically, the growth rate of Staphylococcus aureus is lower than those of Escherichia coli, Salmonella, and Vibrio parahaemolyticus. Therefore, when these food poisoning bacteria are simultaneously cultured in a single culture medium, Escherichia coli and Salmonella grow rapidly, and particularly the growth of Staphylococcus aureus is suppressed (i.e., Staphylococcus aureus does not grow sufficiently). As a result, the detection sensitivity with respect to Staphylococcus aureus may be insufficient.

One or more embodiments of the invention provide a carrier for detecting food poisoning bacteria and a kit for detecting food poisoning bacteria that are used when simultaneously amplifying the amplification target region of Escherichia coli, Salmonella, Staphylococcus aureus, and Vibrio parahaemolyticus by means of multiplex PCR, and implementing detection using a DNA chip on which probes that complementarily binds to the amplified product is immobilized, and make it possible to simultaneously and highly specifically detect Escherichia coli and Vibrio parahaemolyticus that may be non-pathogenic together with enterohemorrhagic Escherichia coli and pathogenic Vibrio parahaemolyticus, and simultaneously and specifically detect Escherichia coli, Salmonella, Staphylococcus aureus, and Vibrio parahaemolyticus in the sample.

One or more embodiments of the invention provide a method for detecting food poisoning bacteria and a PCR reaction mixture for food poisoning bacteria that are used when simultaneously culturing Escherichia coli, Salmonella, Staphylococcus aureus, and Vibrio parahaemolyticus in a single culture medium, and simultaneously amplifying each amplification target region by PCR to detect Escherichia coli, Salmonella, Staphylococcus aureus, and Vibrio parahaemolyticus, and make it possible to advantageously amplify the amplification target region of Staphylococcus aureus, and simultaneously and specifically detect Escherichia coli, Salmonella, Staphylococcus aureus, and Vibrio parahaemolyticus.

A carrier for detecting food poisoning bacteria according to one or more embodiments of the invention is used to simultaneously detect Escherichia coli, Salmonella, Staphylococcus aureus, and Vibrio parahaemolyticus, three or more probes respectively selected from a uridine monophosphate kinase gene (pyrH gene), a verotoxin 1 gene (vtx1 gene), and a verotoxin 2 gene (vtx2 gene) of Escherichia coli, one probe or two or more probes selected from an invasive factor-related gene (invA gene) of Salmonella, one probe or two or more probes selected from a heat shock protein gene (dnaJ gene) of Staphylococcus aureus, and four or more probes respectively selected from virulence regulatory gene (toxR gene), a thermostable direct hemolysin gene (tdh gene), a thermostable direct hemolysin-related hemolysin 1 gene (trh1 gene), and a thermostable direct hemolysin-related hemolysin 2 gene (trh2 gene) of Vibrio parahaemolyticus, being immobilized on the carrier.

A kit for detecting food poisoning bacteria according to one or more embodiments of the invention is used to simultaneously detect Escherichia coli, Salmonella, Staphylococcus aureus, and Vibrio parahaemolyticus, and includes:

the carrier for detecting food poisoning bacteria; and

a PCR reaction mixture that includes:

a pyrH primer set that is used to amplify a DNA fragment that includes the uridine monophosphate kinase gene (pyrH gene) of Escherichia coli, and includes a primer having the base sequence represented by SEQ ID NO: 1 and a primer having the base sequence represented by SEQ ID NO: 2;

a vtx1 primer set that is used to amplify a DNA fragment that includes the verotoxin 1 gene (vtx1 gene) of Escherichia coli, and includes a primer having the base sequence represented by SEQ ID NO: 3 and a primer having the base sequence represented by SEQ ID NO: 4;

a vtx2 primer set that is used to amplify a DNA fragment that includes the verotoxin 2 gene (vtx2 gene) of Escherichia coli, and includes a primer having the base sequence represented by SEQ ID NO: 5 and a primer having the base sequence represented by SEQ ID NO: 6;

an invA primer set that is used to amplify a DNA fragment that includes the invasive factor-related gene (invA gene) of Salmonella, and includes a primer having the base sequence represented by SEQ ID NO: 7 and a primer having the base sequence represented by SEQ ID NO: 8;

a dnaJ primer set that is used to amplify a DNA fragment that includes the heat shock protein gene (dnaJ gene) of Staphylococcus aureus, and includes a primer having the base sequence represented by SEQ ID NO: 9 and a primer having the base sequence represented by SEQ ID NO: 10;

a toxR primer set that is used to amplify a DNA fragment that includes virulence regulatory gene (toxR gene) of Vibrio parahaemolyticus, and includes a primer having the base sequence represented by SEQ ID NO: 11 and a primer having the base sequence represented by SEQ ID NO: 12;

a tdh primer set that is used to amplify a DNA fragment that includes the thermostable direct hemolysin gene (tdh gene) of Vibrio parahaemolyticus, and includes a primer having the base sequence represented by SEQ ID NO: 13 and a primer having the base sequence represented by SEQ ID NO: 14; and

a trh primer set that is used to amplify a DNA fragment that includes the thermostable direct hemolysin-related hemolysin gene (trh gene) of Vibrio parahaemolyticus, and includes a primer having the base sequence represented by SEQ ID NO: 15 and a primer having the base sequence represented by SEQ ID NO: 16,

the concentration of each primer included in the dnaJ primer set in the PCR reaction mixture being higher than the concentration of each primer included in the pyrH primer set, the concentration of each primer included in the vtx1 primer set, the concentration of each primer included in the vtx2 primer set, the concentration of each primer included in the invA primer set, the concentration of each primer included in the toxR primer set, the concentration of each primer included in the tdh primer set, and the concentration of each primer included in the trh primer set in the PCR reaction mixture by a factor of 1.25 or more.

A method for detecting food poisoning bacteria according to one or more embodiments of the invention simultaneously detects Escherichia coli, Salmonella, Staphylococcus aureus, and Vibrio parahaemolyticus, and includes:

an enrichment step that simultaneously enriches Escherichia coli, Salmonella, Staphylococcus aureus, and Vibrio parahaemolyticus using a culture medium in which Escherichia coli, Salmonella, Staphylococcus aureus, and Vibrio parahaemolyticus can be cultured;

an extraction step that extracts genomic DNA of Escherichia coli, Salmonella, Staphylococcus aureus, and Vibrio parahaemolyticus from the culture medium subjected to the enrichment step;

an amplification step that simultaneously amplifies a DNA fragment that includes a uridine monophosphate kinase gene (pyrH gene) of Escherichia coli, a DNA fragment that includes an invasive factor-related gene (invA gene) of Salmonella, a DNA fragment that includes a heat shock protein gene (dnaJ gene) of Staphylococcus aureus, and a DNA fragment that includes virulence regulatory gene (toxR) of Vibrio parahaemolyticus, by means of PCR; and

a detection step that simultaneously detects amplified products obtained by the amplification step by means of electrophoresis or a DNA chip,

the concentration of each primer included in a dnaJ primer set that is used to amplify the DNA fragment that includes the dnaJ gene in a PCR reaction mixture used in the amplification step being higher than the concentration of each primer included in a pyrH primer set that is used to amplify the DNA fragment that includes the pyrH gene, the concentration of each primer included in an invA primer set that is used to amplify the DNA fragment that includes the invA gene, and the concentration of each primer included in a toxR primer set that is used to amplify the DNA fragment that includes the toxR gene in the PCR reaction mixture by a factor of 1.25 or more.

A PCR reaction mixture for food poisoning bacteria according to one or more embodiments of the invention is used to simultaneously amplify Escherichia coli, Salmonella, Staphylococcus aureus, and Vibrio parahaemolyticus by means of PCR, and includes:

a pyrH primer set that is used to amplify a DNA fragment that includes a uridine monophosphate kinase gene (pyrH gene) of Escherichia coli, and includes a primer having the base sequence represented by SEQ ID NO: 1 and a primer having the base sequence represented by SEQ ID NO: 2;

an invA primer set that is used to amplify a DNA fragment that includes an invasive factor-related gene (invA gene) of Salmonella, and includes a primer having the base sequence represented by SEQ ID NO: 7 and a primer having the base sequence represented by SEQ ID NO: 8;

a dnaJ primer set that is used to amplify a DNA fragment that includes a heat shock protein gene (dnaJ gene) of Staphylococcus aureus, and includes a primer having the base sequence represented by SEQ ID NO: 9 and a primer having the base sequence represented by SEQ ID NO: 10; and

a toxR primer set that is used to amplify a DNA fragment that includes virulence regulatory gene (toxR gene) of Vibrio parahaemolyticus, and includes a primer having the base sequence represented by SEQ ID NO: 11 and a primer having the base sequence represented by SEQ ID NO: 12, the concentration of each primer included in the dnaJ primer set in the PCR reaction mixture being 125 nM or more, and the concentration of each primer included in the pyrH primer set, the concentration of each primer included in the invA primer set, and the concentration of each primer included in the toxR primer set in the PCR reaction mixture being respectively 50 to 100 nM.

According to one or more embodiments of the invention, it is possible to simultaneously and highly specifically detect Escherichia coli and Vibrio parahaemolyticus that may be non-pathogenic together with pathogenic Escherichia coli and pathogenic Vibrio parahaemolyticus, and simultaneously and specifically detect Escherichia coli, Salmonella, Staphylococcus aureus, and Vibrio parahaemolyticus in a sample.

According to one or more embodiments of the invention, when simultaneously culturing Escherichia coli, Salmonella, Staphylococcus aureus, and Vibrio parahaemolyticus in a single culture medium, and simultaneously amplifying each amplification target region by PCR to detect Escherichia coli, Salmonella, Staphylococcus aureus, and Vibrio parahaemolyticus, it is possible to advantageously amplify the amplification target region of Staphylococcus aureus, and simultaneously and specifically detect Escherichia coli, Salmonella, Staphylococcus aureus, and Vibrio parahaemolyticus.

### BRIEF DESCRIPTION OF DRAWINGS

FIG. 1 is a view illustrating the presence or absence of a toxin gene in each strain used in Tests 1 and 2 performed in connection with the carrier for detecting food poisoning bacteria and the kit for detecting food poisoning bacteria according to one or more embodiments of the invention.

FIG. 2 is a view illustrating the base sequence (primer sequence) of primer sets corresponding to eight amplification target regions with respect to Escherichia coli, Salmonella, Staphylococcus aureus, and Vibrio parahaemolyticus that are used in connection with the carrier for detecting food poisoning bacteria and the kit for detecting food poisoning bacteria according to one or more embodiments of the invention.

FIG. 3 is a view illustrating the electrophoretic results for the multiplex PCR amplified products when using primer sets corresponding to eight amplification target regions with respect to Escherichia coli, Salmonella, Staphylococcus aureus, and Vibrio parahaemolyticus that are used in connection with the carrier for detecting food poisoning bacteria and the kit for detecting food poisoning bacteria according to one or more embodiments of the invention.

FIG. 4 is a view illustrating the base sequence (probe sequence) (Escherichia coli and Salmonella) of the probes used in connection with the carrier for detecting food poisoning bacteria and the kit for detecting food poisoning bacteria according to one or more embodiments of the invention.

FIG. 5 is a view illustrating the base sequence (probe sequence) (Staphylococcus aureus and Vibrio parahaemolyticus) of the probes used in connection with the carrier for detecting food poisoning bacteria and the kit for detecting food poisoning bacteria according to one or more embodiments of the invention.

FIG. 6 is a view illustrating the food poisoning bacterium detection results (fluorescence intensity) (Escherichia coli and Salmonella) obtained using the carrier for detecting food poisoning bacteria and the kit for detecting food poisoning bacteria according to one or more embodiments of the invention.

FIG. 7 is a view illustrating the food poisoning bacterium detection results (fluorescence intensity) (Staphylococcus aureus and Vibrio parahaemolyticus) obtained using the carrier for detecting food poisoning bacteria and the kit for detecting food poisoning bacteria according to one or more embodiments of the invention.

FIG. 8 is a view illustrating the electrophoretic results for the determination target species obtained using the primer set (pyrH primer set) for amplifying a uridine monophosphate kinase gene region that is used in connection with the kit for detecting food poisoning bacteria according to one or more embodiments of the invention.

FIG. 9 is a view illustrating the detection results (fluorescence intensity and S/N ratio) for a plurality of probes (pyrH probes) selected from a uridine monophosphate kinase gene region that are used in connection with the carrier for detecting food poisoning bacteria according to one or more embodiments of the invention.

FIG. 10 is a view illustrating presence or absence of a toxin gene included in the Vibrio parahaemolyticus strains used in Test 4 performed in connection with the carrier for detecting food poisoning bacteria and the kit for detecting food poisoning bacteria according to one or more embodiments of the invention.

FIG. 11 is a view illustrating the primer sets (trh primer sets) for amplifying a thermostable direct hemolysin-related hemolysin gene (trh) region used in connection with the kit for detecting food poisoning bacteria according to one or more embodiments of the invention, and the detection results (fluorescence intensity) obtained using the probe (trh1 probe) selected from the thermostable direct hemolysin-related hemolysin 1 gene (trh1 gene) in the thermostable direct hemolysin-related hemolysin gene (trh) region and the probe (trh2 probe) selected from the thermostable direct hemolysin-related hemolysin 2 gene (trh2 gene) in the thermostable direct hemolysin-related hemolysin gene (trh) region used in connection with the carrier for detecting food poisoning bacteria according to one or more embodiments of the invention.

FIG. 12 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which gyoza was added, and multiplex PCR was performed in a state in which the concentration of the primers for Staphylococcus aureus was fixed, and the concentrations of the primers for Escherichia coli, Salmonella, and Vibrio parahaemolyticus were changed.

FIG. 13 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which precut vegetables were added, and multiplex PCR was performed in a state in which the concentration of the primers for Staphylococcus aureus was fixed, and the concentrations of the primers for Escherichia coli, Salmonella, and Vibrio parahaemolyticus were changed.

FIG. 14 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which uncured ham was added, and multiplex PCR was performed in a state in which the concentration of the primers for Staphylococcus aureus was fixed, and the concentrations of the primers for Escherichia coli, Salmonella, and Vibrio parahaemolyticus were changed.

FIG. 15 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which fish meat sausage was added, and multiplex PCR was performed in a state in which the concentration of the primers for Staphylococcus aureus was fixed, and the concentrations of the primers for Escherichia coli, Salmonella, and Vibrio parahaemolyticus were changed.

FIG. 16 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which gyoza was added, and multiplex PCR was performed in a state in which the concentration of the primers for Staphylococcus aureus was changed, and the concentrations of the primers for Escherichia coli, Salmonella, and Vibrio parahaemolyticus were fixed.

FIG. 17 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which precut vegetables were added, and multiplex PCR was performed in a state in which the concentration of the primers for Staphylococcus aureus was changed, and the concentrations of the primers for Escherichia coli, Salmonella, and Vibrio parahaemolyticus were fixed.

FIG. 18 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which uncured ham was added, and multiplex PCR was performed in a state in which the concentration of the primers for Staphylococcus aureus was changed, and the concentrations of the primers for Escherichia coli, Salmonella, and Vibrio parahaemolyticus were fixed.

FIG. 19 is a view illustrating the amount of amplified product (on a bacterial species basis) when Escherichia coli, Salmonella, Staphylococcus aureus, and Vibrio parahaemolyticus (that are used in connection with the carrier for detecting food poisoning bacteria and the kit for detecting food poisoning bacteria according to one or more embodiments of the invention) were simultaneously cultured using a culture medium to which fish meat sausage was added, and multiplex PCR was performed in a state in which the concentration of the primers for Staphylococcus aureus was changed, and the concentrations of the primers for Escherichia coli, Salmonella, and Vibrio parahaemolyticus were fixed.

### DETAILED DESCRIPTION OF THE INVENTION

A carrier for detecting food poisoning bacteria, a kit for detecting food poisoning bacteria, a method for detecting food poisoning bacteria, and a PCR reaction mixture for food poisoning bacteria according to one or more embodiments of the invention are described in detail below.

A carrier for detecting food poisoning bacteria according to one or more embodiments of the invention is used to simultaneously detect Escherichia coli, Salmonella, Staphylococcus aureus, and Vibrio parahaemolyticus, three or more probes respectively selected from a uridine monophosphate kinase gene (pyrH gene), a verotoxin 1 gene (vtx1 gene), and a verotoxin 2 gene (vtx2 gene) of Escherichia coli, one probe or two or more probes selected from an invasive factor-related gene (invA gene) of Salmonella, one probe or two or more probes selected from a heat shock protein gene (dnaJ gene) of Staphylococcus aureus, and four or more probes respectively selected from virulence regulatory gene (toxR gene), a thermostable direct hemolysin gene (tdh gene), a thermostable direct hemolysin-related hemolysin 1 gene (trh1 gene), and a thermostable direct hemolysin-related hemolysin 2 gene (trh2 gene) of Vibrio parahaemolyticus, being immobilized on the carrier.

### Detection Target Bacteria

Escherichia coli

Escherichia coli is a gram-negative facultative anaerobic bacillus. Escherichia coli is one type of intestinal bacteria. Most of them do not have pathogenicity, and are harmless to humans. In the field of public health, however, a wide variety of Escherichia coli may be detected as a hygienic index (e.g., an index with respect to fecal contamination) irrespective of pathogenicity.

A method for detecting food poisoning bacteria according to one or more embodiments of the invention makes it possible to detect the presence or absence of Escherichia coli including pathogenic Escherichia coli and non-pathogenic Escherichia coli in a sample by amplifying a uridine monophosphate kinase gene (pyrH gene) (i.e., amplification target region) that is commonly included in the genomic DNA of Escherichia coli by means of PCR, and detecting the amplified product.

Some of Escherichia coli cause stomachache, diarrhea, and the like, and are generally referred to as “pathogenic Escherichia coli”. In particular, enterohemorrhagic Escherichia coli (EHEC) (e.g., O157) may include either or both of a verotoxin 1 gene (vtx1 gene) and a verotoxin 2 gene (vtx2 gene) as a gene that produces toxin.

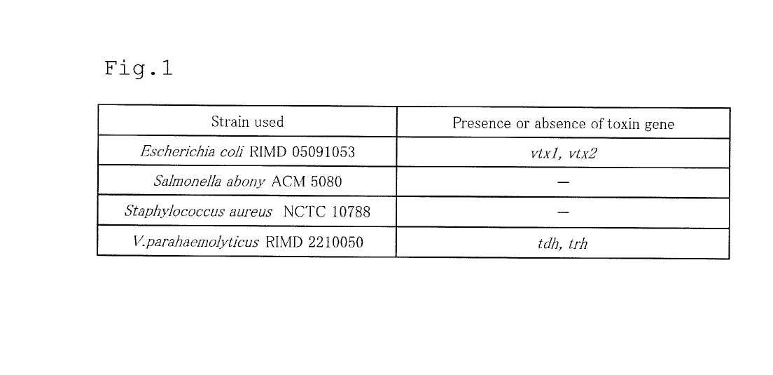

For example, some strains are known to include a verotoxin gene (see FIG. 1). In the examples described later, a pyrH gene, a vtx1 gene, and a vtx2 gene were simultaneously detected using these strains.

### Salmonella

Salmonella is a gram-negative facultative anaerobic bacillus, and is one type of intestinal bacteria. Some of them cause infectious food poisoning. The method for detecting food poisoning bacteria according to one or more embodiments of the invention makes it possible to detect the presence or absence of Salmonella in a sample by amplifying an invasive factor-related gene (invA gene) (i.e., amplification target region) that is commonly included in the genomic DNA of Salmonella by means of PCR, and detecting the amplified product.

Staphylococcus aureus

Staphylococcus aureus is one type of Staphylococcus, and is a gram-positive facultative anaerobic micrococcus. Staphylococcus aureus resides on human skin and in the nasal cavity and intestines, and may cause diseases even in healthy people. However, the toxicity of Staphylococcus aureus is normally weak when the number thereof is small. Staphylococcus aureus may cause food poisoning, and various infectious diseases such as epidermal infections, pneumonia, and meningitis. The method for detecting food poisoning bacteria according to one or more embodiments of the invention makes it possible to detect the presence or absence of Staphylococcus aureus in a sample by amplifying a heat shock protein gene (dnaJ gene) (i.e., amplification target region) that is commonly included in the genomic DNA of Staphylococcus aureus by means of PCR, and detecting the amplified product.

Vibrio parahaemolyticus

Vibrio parahaemolyticus is a gram-negative halophilic bacillus. Vibrio parahaemolyticus is mainly found in seawater. Pathogenic Vibrio parahaemolyticus causes a human to develop food poisoning through infection. Non-pathogenic Vibrio parahaemolyticus is also present. In the field of public health, however, a wide variety of Vibrio parahaemolyticus may be detected as a hygienic index irrespective of pathogenicity.

The method for detecting food poisoning bacteria according to one or more embodiments of the invention makes it possible to detect the presence or absence of Vibrio parahaemolyticus including pathogenic Vibrio parahaemolyticus and non-pathogenic Vibrio parahaemolyticus in a sample by amplifying virulence regulatory gene (toxR) (i.e., amplification target region) that is commonly included in the genomic DNA of Vibrio parahaemolyticus by means of PCR, and detecting the amplified product.

Pathogenic Vibrio parahaemolyticus may include either or both of a thermostable direct hemolysin gene (tdh gene) and a thermostable direct hemolysin-related hemolysin gene (trh gene).

For example, some pathogenic Vibrio parahaemolyticus strains are known to include a toxin gene (see FIG. 1). The thermostable direct hemolysin-related hemolysin genes (trh genes) are classified into thermostable direct hemolysin-related hemolysin 1 gene (trh1 gene) and a thermostable direct hemolysin-related hemolysin 2 gene (trh2 gene). In the examples described later, a toxR gene, a tdh gene, and a trh gene were simultaneously detected using these strains, and a trh1 gene and a trh2 gene were identified.

Note that it is also important to detect non-pathogenic Escherichia coli, Salmonella, Staphylococcus aureus, and Vibrio parahaemolyticus from the viewpoint of a hygienic index, and the term “food poisoning bacteria” is used herein to also include non-pathogenic Escherichia coli, Salmonella, Staphylococcus aureus, and Vibrio parahaemolyticus for convenience.

### Culture Medium

A culture medium that includes a peptone, a yeast extract, magnesium sulfate, and sodium chloride may be used as a culture medium for simultaneously culturing the four types of food poisoning bacteria used for the carrier for detecting food poisoning bacteria according to one or more embodiments of the invention and a kit for detecting food poisoning bacteria according to one or more embodiments of the invention.

Since the growth rate of Staphylococcus aureus is lower than those of Escherichia coli, Salmonella, and Vibrio parahaemolyticus, it is difficult to simultaneously culture Escherichia coli, Salmonella, Staphylococcus aureus, and Vibrio parahaemolyticus in a single culture medium (i.e., it may be impossible to sufficiently grow Staphylococcus aureus). However, it is possible to promote the growth of Staphylococcus aureus by utilizing the above culture medium.

The kit for detecting food poisoning bacteria according to one or more embodiments of the invention is characterized in that the concentration of each primer of the primer set for amplifying the amplification target region of the genomic DNA of Staphylococcus aureus, and the concentration of each primer of the primer set for amplifying the amplification target region of the genomic DNA of Escherichia coli, Salmonella, and Vibrio parahaemolyticus, are appropriately adjusted as described later in order to improve the detection accuracy with respect to Staphylococcus aureus. This makes it possible to simultaneously amplify the amplification target regions of Escherichia coli, Salmonella, Staphylococcus aureus, and Vibrio parahaemolyticus in a well-balanced manner to obtain amplified products that can be detected.

The culture medium may include a phosphate (0.35% w/v disodium hydrogen phosphate+0.15% w/v potassium dihydrogen phosphate) as an additional component. The culture medium may include a further additional component.

The pH of the culture medium may be adjusted to 6.5 to 7.5. When the pH of the culture medium is within the above range, it is possible to simultaneously grow Escherichia coli, Salmonella, Staphylococcus aureus, and Vibrio parahaemolyticus in an advantageous manner. If the pH of the culture medium is lower or higher than the above range, the growth of Staphylococcus aureus may be insufficient.

The culture medium may be produced by mixing a peptone, a yeast extract, magnesium sulfate, sodium chloride, disodium hydrogen phosphate, and potassium dihydrogen phosphate, adjusting the pH of the mixture to 7.0, and sterilizing the mixture at 121° C. for 15 minutes using an autoclave, for example.

### Carrier for Detecting Food Poisoning Bacteria

The carrier (DNA chip) for detecting food poisoning bacteria according to one or more embodiments of the invention may have a configuration in which at least one probe having a base sequence among the base sequences respectively represented by SEQ ID NO: 17 to 19 is immobilized on the carrier as the probe (pyrH probe) selected from the uridine monophosphate kinase gene (pyrH gene) region of Escherichia coli. The carrier for detecting food poisoning bacteria according to one or more embodiments of the invention may have a configuration in which at least one probe having the base sequence represented by SEQ ID NO: 18 or the base sequence represented by SEQ ID NO: 19 is immobilized on the carrier from the viewpoint of specificity. The carrier for detecting food poisoning bacteria according to one or more embodiments of the invention may have a configuration in which two or more probes having a base sequence among the base sequences respectively represented by SEQ ID NO: 17 to 19 are immobilized on the carrier. It is possible to reduce the possibility that a false-positive determination result is obtained, by combining probes having excellent specificity.

The carrier for detecting food poisoning bacteria according to one or more embodiments of the invention may have a configuration in which at least one probe having a base sequence among the base sequences respectively represented by SEQ ID NO: 20 to 27 is immobilized on the carrier as the probe (vtx1 probe) selected from the verotoxin 1 gene (vtx1 gene) region of enterohemorrhagic Escherichia coli. The carrier for detecting food poisoning bacteria according to one or more embodiments of the invention may have a configuration in which at least one probe having a base sequence among the base sequences respectively represented by SEQ ID NO: 28 to 32 is immobilized on the carrier as the probe (vtx2 probe) selected from the verotoxin 2 gene (vtx2 gene) region of enterohemorrhagic Escherichia coli.

When the vtx1 probe and the vtx2 probe are immobilized on the carrier for detecting food poisoning bacteria according to one or more embodiments of the invention in addition to the pyrH probe, it is possible to simultaneously and specifically detect Escherichia coli (including enterohemorrhagic Escherichia coli) and enterohemorrhagic Escherichia coli having either or both of a verotoxin 1 gene and a verotoxin 2 gene.

The carrier for detecting food poisoning bacteria according to one or more embodiments of the invention may have a configuration in which at least one probe having a base sequence among the base sequences respectively represented by SEQ ID NO: 33 to 37 is immobilized on the carrier as the probe (invA probe) selected from the invasive factor-related gene (invA gene) region of Salmonella.

The carrier for detecting food poisoning bacteria according to one or more embodiments of the invention may have a configuration in which at least one probe having a base sequence among the base sequences respectively represented by SEQ ID NO: 38 to 40 is immobilized on the carrier as the probe (dnaJ probe) selected from the heat shock protein gene (dnaJ gene) region of Staphylococcus aureus.

The carrier for detecting food poisoning bacteria according to one or more embodiments of the invention may have a configuration in which at least one probe having a base sequence among the base sequences respectively represented by SEQ ID NO: 41 to 44 is immobilized on the carrier as the probe (toxR probe) selected from virulence regulatory gene (toxR gene) region of Vibrio parahaemolyticus.

The carrier for detecting food poisoning bacteria according to one or more embodiments of the invention may have a configuration in which at least one probe having a base sequence among the base sequences respectively represented by SEQ ID NO: 45 to 49 is immobilized on the carrier as the probe (tdh probe) selected from the thermostable direct hemolysin gene (tdh gene) region of pathogenic Vibrio parahaemolyticus.

The carrier for detecting food poisoning bacteria according to one or more embodiments of the invention may have a configuration in which at least one probe having a base sequence among the base sequences respectively represented by SEQ ID NO: 50 to 53 is immobilized on the carrier as the probe (trh1 probe) selected from the thermostable direct hemolysin-related hemolysin 1 gene included in the thermostable direct hemolysin-related hemolysin gene (trh gene) region of pathogenic Vibrio parahaemolyticus. The carrier for detecting food poisoning bacteria according to one or more embodiments of the invention may have a configuration in which at least one probe having the base sequence represented by SEQ ID NO: 54 or the base sequence represented by SEQ ID NO: 55 is immobilized on the carrier as the probe (trh2 probe) selected from the thermostable direct hemolysin-related hemolysin 2 gene included in the thermostable direct hemolysin-related hemolysin gene (trh gene) region of pathogenic Vibrio parahaemolyticus.

When the carrier for detecting food poisoning bacteria according to one or more embodiments of the invention has the above configuration, it is possible to simultaneously and specifically detect Vibrio parahaemolyticus (including pathogenic Vibrio parahaemolyticus) and pathogenic Vibrio parahaemolyticus. It is also possible to determine whether pathogenic Vibrio parahaemolyticus has either or both of a thermostable direct hemolysin gene (tdh gene) and a thermostable direct hemolysin-related hemolysin gene (trh gene). It is also possible to determine whether pathogenic Vibrio parahaemolyticus has either or both of a thermostable direct hemolysin-related hemolysin 1 gene and a thermostable direct hemolysin-related hemolysin 2 gene as the thermostable direct hemolysin-related hemolysin gene (trh gene).

It is possible to reduce the number of primers used for multiplex PCR, and implement an improvement in accuracy and a reduction in cost by detecting a thermostable direct hemolysin-related hemolysin 1 gene and a thermostable direct hemolysin-related hemolysin 2 gene using a dedicated probe having high special specificity after obtaining an amplified product using an identical primer, instead of detecting a thermostable direct hemolysin-related hemolysin 1 gene and a thermostable direct hemolysin-related hemolysin 2 gene based on the size of the amplified product using a dedicated primer.

Each probe used for the carrier for detecting food poisoning bacteria according to one or more embodiments of the invention is not limited to the above base sequence. Each probe may be a probe having the corresponding base sequence wherein one base or a plurality of bases are deleted, substituted, or inserted (added). Each probe may be a probe that includes a nucleic acid fragment that hybridizes to a nucleic acid fragment having a base sequence complementary to the corresponding base sequence under stringent conditions. It is also possible to use a probe having a base sequence complementary to that of such a probe.

The term “stringent conditions” used herein refers to conditions under which a specific hybrid is formed, but a non-specific hybrid is not formed. For example, the stringent conditions may be conditions under which DNA having high homology (for example, 90% or more, and 95% or more) with DNA having a sequence among the sequences respectively represented by SEQ ID NO: 17 to 55 hybridizes to DNA having a base sequence complementary to that of DNA having the sequence among the sequences respectively represented by SEQ ID NO: 17 to 55. The stringent conditions normally refer to conditions under which hybridization occurs at a temperature lower than the melting temperature (Tm) of a perfect hybrid by about 5° C. to about 30° C. (for example, about 10° C. to about 25° C.). For example, the conditions described in J. Sambrook et al., Molecular Cloning, A Laboratory Manual, Second Edition, Cold Spring Harbor Laboratory Press (1989) (particularly the conditions described in section 11.45 “Conditions for Hybridization of Oligonucleotide Probes”) may be used as the stringent conditions.

### Method for Detecting Food Poisoning Bacteria

The method for detecting food poisoning bacteria according to one or more embodiments of the invention may include an enrichment step that enriches food poisoning bacteria, an extraction step that extracts genomic DNA from the food poisoning bacteria, an amplification step that amplifies the DNA fragment in the amplification target region of the genomic DNA, and a detection step that detects the resulting amplified products.

In the enrichment step, Escherichia coli, Salmonella, Staphylococcus aureus, and Vibrio parahaemolyticus are simultaneously enriched using a culture medium in which Escherichia coli, Salmonella, Staphylococcus aureus, and Vibrio parahaemolyticus can be cultured. Specifically, Escherichia coli, Salmonella, Staphylococcus aureus, and Vibrio parahaemolyticus in a sample are grown using the culture medium until a bacterial count (e.g., 10⁵ cfu/mL or more) appropriate for amplification using PCR is reached. Examples of the sample include food, a clinical sample (feces or vomit), and the like. When food is used as the sample, it is possible to grow the food poisoning bacteria up to an appropriate bacterial count by adding 25 g of the food to 225 mL of the culture medium, and culturing the food poisoning bacteria at 37° C. for about 20 hours.

In the extraction step, the genomic DNA of Escherichia coli, Salmonella, Staphylococcus aureus, and Vibrio parahaemolyticus is extracted from the culture medium obtained by the enrichment step. The genomic DNA may be extracted using an arbitrary method. For example, the culture medium is collected, and centrifuged. After removing the supernatant liquid, a lysozyme solution suitable for bacteriolysis of gram-positive bacteria is added to the precipitate, and a bacteriolysis treatment is performed. Proteolysis and column purification are performed to obtain a DNA extract, which is used as a sample to be amplified by PCR.

In the amplification step, a DNA fragment that includes the uridine monophosphate kinase gene (pyrH gene) of Escherichia coli, a DNA fragment that includes the verotoxin 1 gene (vtx1 gene) of Escherichia coli, a DNA fragment that includes the verotoxin 2 gene (vtx2 gene) of Escherichia coli, a DNA fragment that includes the invasive factor-related gene (invA gene) of Salmonella, a DNA fragment that includes the heat shock protein gene (dnaJ gene) of Staphylococcus aureus, a DNA fragment that includes virulence regulatory gene (toxR) of Vibrio parahaemolyticus, a DNA fragment that includes the thermostable direct hemolysin gene (tdh gene) of Vibrio parahaemolyticus, and a DNA fragment that includes the thermostable direct hemolysin-related hemolysin gene (trh gene) of Vibrio parahaemolyticus are simultaneously amplified by multiplex PCR.

In the amplification step, it is also possible to simultaneously amplify a DNA fragment that includes the uridine monophosphate kinase gene (pyrH gene) of Escherichia coli, a DNA fragment that includes the invasive factor-related gene (invA gene) of Salmonella, a DNA fragment that includes the heat shock protein gene (dnaJ gene) of Staphylococcus aureus, and a DNA fragment that includes virulence regulatory gene (toxR) of Vibrio parahaemolyticus, by multiplex PCR.

In the detection step, the amplified products obtained by the amplification step are simultaneously detected using the carrier for detecting food poisoning bacteria according to one or more embodiments of the invention.

In the detection step, the amplified products obtained by the amplification step may be simultaneously detected by means of electrophoresis or a DNA chip.

Specifically, it is also possible to subject the amplified products (PCR amplified products) obtained by PCR to electrophoresis to check whether or not the amplified products have been obtained due to each primer set according to one or more embodiments of the invention, and determine (detect) the presence or absence of each type of food poisoning bacteria in the sample. Electrophoresis may be implemented using a common method (e.g., agarose gel electrophoresis, polyacrylamide gel electrophoresis, or microchip electrophoresis).

The amplification step and the detection step included in the method for detecting food poisoning bacteria according to one or more embodiments of the invention are further described below in connection with the kit for detecting food poisoning bacteria.

### Kit for Detecting Food Poisoning Bacteria

The kit for detecting food poisoning bacteria according to one or more embodiments of the invention includes the carrier for detecting food poisoning bacteria described above, and a PCR reaction mixture.

The PCR reaction mixture may include a pyrH primer set that is used to amplify a DNA fragment that includes the uridine monophosphate kinase gene (pyrH gene) of Escherichia coli, and includes a primer having the base sequence represented by SEQ ID NO: 1 and a primer having the base sequence represented by SEQ ID NO: 2, a vtx1 primer set that is used to amplify a DNA fragment that includes the verotoxin 1 gene (vtx1 gene) of Escherichia coli, and includes a primer having the base sequence represented by SEQ ID NO: 3 and a primer having the base sequence represented by SEQ ID NO: 4, a vtx2 primer set that is used to amplify a DNA fragment that includes the verotoxin 2 gene (vtx2 gene) of Escherichia coli, and includes a primer having the base sequence represented by SEQ ID NO: 5 and a primer having the base sequence represented by SEQ ID NO: 6, an invA primer set that is used to amplify a DNA fragment that includes the invasive factor-related gene (invA gene) of Salmonella, and includes a primer having the base sequence represented by SEQ ID NO: 7 and a primer having the base sequence represented by SEQ ID NO: 8, a dnaJ primer set that is used to amplify a DNA fragment that includes the heat shock protein gene (dnaJ gene) of Staphylococcus aureus, and includes a primer having the base sequence represented by SEQ ID NO: 9 and a primer having the base sequence represented by SEQ ID NO: 10, a toxR primer set that is used to amplify a DNA fragment that includes virulence regulatory gene (toxR gene) of Vibrio parahaemolyticus, and includes a primer having the base sequence represented by SEQ ID NO: 11 and a primer having the base sequence represented by SEQ ID NO: 12, a tdh primer set that is used to amplify a DNA fragment that includes the thermostable direct hemolysin gene (tdh gene) of Vibrio parahaemolyticus, and includes a primer having the base sequence represented by SEQ ID NO: 13 and a primer having the base sequence represented by SEQ ID NO: 14, and a trh primer set that is used to amplify a DNA fragment that includes the thermostable direct hemolysin-related hemolysin gene (trh gene) of Vibrio parahaemolyticus, and includes a primer having the base sequence represented by SEQ ID NO: 15 and a primer having the base sequence represented by SEQ ID NO: 16.

The PCR reaction mixture may include a pyrH primer set that is used to amplify a DNA fragment that includes the uridine monophosphate kinase gene (pyrH gene) of Escherichia coli, and includes a primer having the base sequence represented by SEQ ID NO: 1 and a primer having the base sequence represented by SEQ ID NO: 2, an invA primer set that is used to amplify a DNA fragment that includes the invasive factor-related gene (invA gene) of Salmonella, and includes a primer having the base sequence represented by SEQ ID NO: 7 and a primer having the base sequence represented by SEQ ID NO: 8, a dnaJ primer set that is used to amplify a DNA fragment that includes the heat shock protein gene (dnaJ gene) of Staphylococcus aureus, and includes a primer having the base sequence represented by SEQ ID NO: 9 and a primer having the base sequence represented by SEQ ID NO: 10, and a toxR primer set that is used to amplify a DNA fragment that includes virulence regulatory gene (toxR gene) of Vibrio parahaemolyticus, and includes a primer having the base sequence represented by SEQ ID NO: 11 and a primer having the base sequence represented by SEQ ID NO: 12.

Each primer included in the PCR reaction mixture is not limited to the above base sequence. Each primer may be a primer having the corresponding base sequence wherein one base or a plurality of bases are deleted, substituted, or inserted (added).

The PCR reaction mixture may include an additional component that is commonly used for a PCR reaction mixture. Specifically, the PCR reaction mixture may include a buffer, a nucleic acid substrate, a nucleic acid polymerase (e.g., Ex Taq), a labeling component (e.g., Cy5), the DNA of the sample, water, and the like.

A region (part) of the genomic DNA included in the sample is amplified using the PCR reaction mixture utilizing a nucleic acid amplification system (e.g., thermal cycler). Specifically, when the sample includes genomic DNA having the amplification target region that is amplified by each primer set, each amplification target region can be simultaneously and specifically amplified by utilizing the PCR reaction mixture that includes each primer set described above.

The concentration of each primer included in the dnaJ primer set that is used to amplify a DNA fragment that includes the heat shock protein gene (dnaJ gene) of Staphylococcus aureus in the PCR reaction mixture may be higher than the concentration of each primer included in the pyrH primer set that is used to amplify a DNA fragment that includes the uridine monophosphate kinase gene (pyrH gene) of Escherichia coli, the concentration of each primer included in the invA primer set that is used to amplify a DNA fragment that includes the invasive factor-related gene (invA gene) of Salmonella, and the concentration of each primer included in the toxR primer set that is used to amplify a DNA fragment that includes virulence regulatory gene (toxR gene) of Vibrio parahaemolyticus in the PCR reaction mixture by a factor of 1.25 or more, for example, 1.25 to 3.5, and 1.5 to 3.

The concentration of each primer included in the dnaJ primer set in the PCR reaction mixture may be 125 nM or more, and the concentration of each primer included in the pyrH primer set, the concentration of each primer included in the invA primer set, and the concentration of each primer included in the toxR primer set in the PCR reaction mixture may be 50 to 100 nM. The concentration of each primer included in the dnaJ primer set in the PCR reaction mixture may be 125 to 175 nM, and the concentration of each primer included in the pyrH primer set, the concentration of each primer included in the invA primer set, and the concentration of each primer included in the toxR primer set in the PCR reaction mixture may be 50 to 100 nM. The concentration of each primer included in the dnaJ primer set in the PCR reaction mixture may be 150 nM, and the concentration of each primer included in the pyrH primer set, the concentration of each primer included in the invA primer set, and the concentration of each primer included in the toxR primer set in the PCR reaction mixture may be 50 to 100 nM.

When the PCR reaction mixture for food poisoning bacteria according to one or more embodiments of the invention includes each primer included in the dnaJ primer set, each primer included in the pyrH primer set, each primer included in the invA primer set, and each primer included in the toxR primer set in a ratio within the above range, it is possible to simultaneously amplify the amplification target region of Staphylococcus aureus together with the amplification target region of Escherichia coli, the amplification target region of Salmonella, and the amplification target region of Vibrio parahaemolyticus in an advantageous manner.

It is possible to determine (detect) the presence or absence of food poisoning bacteria in the sample by adding the amplified products (PCR amplified products) obtained by PCR dropwise to the carrier for detecting food poisoning bacteria according to one or more embodiments of the invention, and detecting the label of the amplified product that has hybridized to each probe.

More specifically, a specific buffer is mixed with the PCR amplified products, and the mixture is added dropwise to the carrier for detecting food poisoning bacteria according to one or more embodiments of the invention. After allowing the carrier to stand at 45° C. for 1 hour, the PCR amplified products that have not hybridized and the like are washed away from the carrier using a specific buffer. The label is detected from the carrier using a label detection system.

The label may be detected using a common label detection system (e.g., fluorescence scanning device). For example, the label may be detected by measuring the fluorescence intensity of the amplified products using a label detection system “BIOSHOT (registered trademark)” manufactured by Toyo Kohan Co., Ltd. It is also possible to calculate the S/N ratio (signal to noise ratio (median fluorescence intensity value−background value)+background value) as the measurement result. Specifically, whether the measurement result is positive or negative can be accurately determined based on the S/N ratio. It is normally determined that the measurement result is positive when the S/N ratio is 3 or more. Note that the label is not limited to fluorescence. Another label may also be used.

As described above, it is possible to simultaneously and highly specifically detect Escherichia coli and Vibrio parahaemolyticus that may be non-pathogenic together with pathogenic Escherichia coli and pathogenic Vibrio parahaemolyticus, advantageously amplify the target gene of Staphylococcus aureus, and simultaneously and specifically detect Escherichia coli, Salmonella, Staphylococcus aureus, and Vibrio parahaemolyticus in a sample, by utilizing the carrier for detecting food poisoning bacteria and the kit for detecting food poisoning bacteria according to one or more embodiments of the invention.

It is also possible to determine (detect) whether pathogenic Vibrio parahaemolyticus has either or both of a thermostable direct hemolysin gene (tdh gene) and a thermostable direct hemolysin-related hemolysin gene (trh gene), and determine (detect) whether pathogenic Vibrio parahaemolyticus has either or both of a thermostable direct hemolysin-related hemolysin 1 gene (trh1 gene) and a thermostable direct hemolysin-related hemolysin 2 gene (trh2 gene) as the thermostable direct hemolysin-related hemolysin gene (trh gene).

It is possible to simultaneously culture Escherichia coli, Salmonella, Staphylococcus aureus, and Vibrio parahaemolyticus in a single culture medium, and simultaneously amplify each amplification target region by multiplex PCR, and simultaneously and specifically detect Escherichia coli, Salmonella, Staphylococcus aureus, and Vibrio parahaemolyticus by utilizing the method for detecting food poisoning bacteria and the PCR reaction mixture for food poisoning bacteria according to one or more embodiments of the invention.

### EXAMPLES

### Test 1: Electrophoretic Identification of Eight Types of Amplified Products Simultaneously Amplified by PCR

A peptone, a yeast extract, magnesium sulfate, sodium chloride, disodium hydrogen phosphate, and potassium dihydrogen phosphate were mixed in the amounts listed below. The mixture was dissolved in distilled water to adjust the pH of the mixture to 7.0. The resulting culture medium was sterilized at 121° C. for 15 minutes using an autoclave.
(Amount per L)
Peptone: 30 g
Yeast extract: 5 g
Magnesium sulfate heptahydrate: 0.5 g
Sodium chloride: 15 g
Disodium hydrogen phosphate: 3.5 g
Potassium dihydrogen phosphate: 1.5 g

The Escherichia coli, Salmonella, Staphylococcus aureus, and Vibrio parahaemolyticus strains illustrated in FIG. 1 were inoculated into the culture medium respectively in an amount of about 10 to 50 cfu, simultaneously cultured at 37° C. for 20 hours, and DNA was extracted from the culture.

The strains of these food poisoning bacteria were transferred from the following organizations.
RIMD: Research Institute for Microbial Diseases, Osaka University (Japan)
ACM: Australian Collection of Microorganisms (Australia)
NCTC: National Collection of Type Cultures (U.K.)

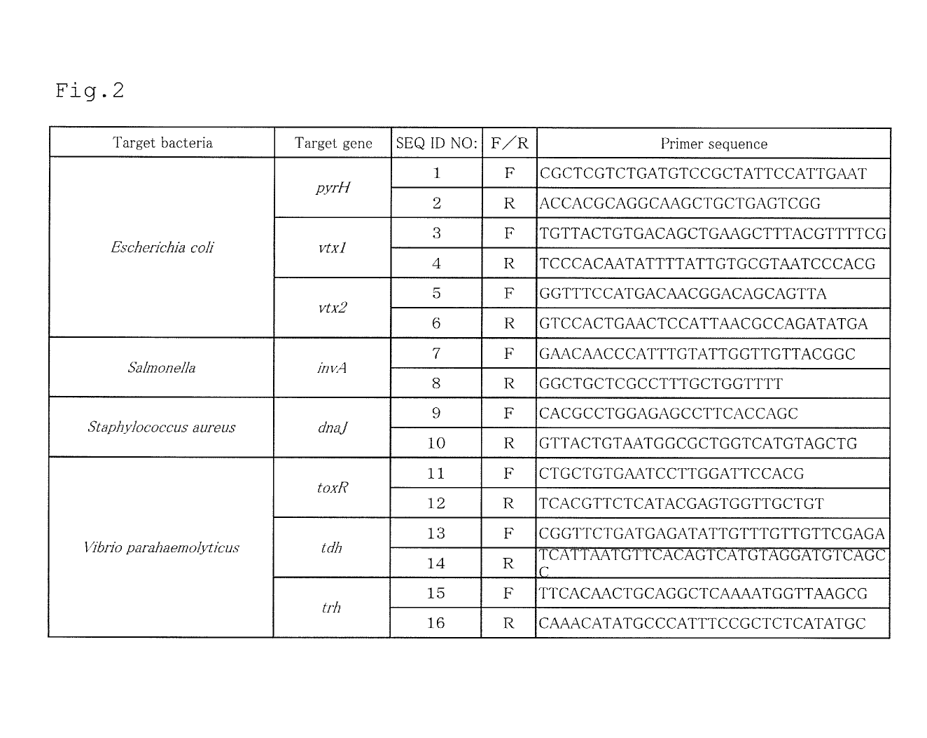

The amplification target regions of the respective food poisoning bacteria were simultaneously amplified by multiplex PCR using the eight primer sets illustrated in FIG. 2, and whether or not the resulting amplified products can be identified was determined. Specifically, the following operation was performed.

1 mL of the culture was collected, and centrifuged at 5000×g for 10 minutes. After removing the supernatant liquid, a lysozyme solution (concentration: 20 mg/mL) (20 mM Tris-HCl, pH: 8.0/2 mM EDTA, 1.2% Triton X-100) was added to the precipitate, and the mixture was subjected to a bacteriolysis treatment at 37° C. for 30 minutes. The resulting mixture was subjected to column purification using a DNeasy Blood & Tissue Kit (manufactured by Qiagen) to obtain a DNA extract. The DNA extract was used as a sample subjected to PCR.

A PCR reaction mixture having the composition shown below was prepared. Each primer was synthesized by Sigma-Aldrich Co. LLC. on commission, and the other reagents were products manufactured by Takara Bio Inc. Note that Vibrio parahaemolyticus is abbreviated as “Vibrio”.
Buffer (10×Ex Taq buffer): 2.0 μL
Nucleic acid substrate (dNTP Mixture): 1.6 μL
Escherichia coli pyrH amplification F primer (Cy5-modified (5′-terminal)): 0.15 μL
Escherichia coli pyrH amplification R primer: 0.15 μL
Escherichia coli vtx1 amplification F primer: 0.15 μL
Escherichia coli vtx1 amplification R primer (Cy5-modified (5′-terminal)): 0.15 μL
Escherichia coli vtx2 amplification F primer: 0.15 μL
Escherichia coli vtx2 amplification R primer (Cy5-modified (5′-terminal)): 0.15 μL
Salmonella invA F primer: 0.15 μL
Salmonella invA R primer (Cy5-modified (5′-terminal)): 0.15 μL
Staphylococcus aureus dnaJ F primer: 0.3 μL
Staphylococcus aureus dnaJ R primer (Cy5-modified (5′-terminal)): 0.3 μL
Vibrio toxR amplification F primer: 0.15 μL
Vibrio toxR amplification R primer (Cy5-modified (5′-terminal)): 0.15 μL
Vibrio tdh amplification F primer: 0.15 μL
Vibrio tdh amplification R primer (Cy5-modified (5′-terminal)): 0.15 μL
Vibrio trh amplification F primer: 0.15 μL
Vibrio trh amplification R primer (Cy5-modified (5′-terminal)): 0.15 μL
TaKaRa Ex Taq Hot Start Version: 0.2 μL
DNA in sample: 1.0 μL
Sterilized water: 12.5 μL
(Total amount: 20 μL)

PCR gene amplification was performed using a system “Thermal Cycler ep gradient” (manufactured by Eppendorf). The reaction conditions are listed below.
(1) 95° C., 2 min
(2) 95° C., 10 sec (denaturation step)
(3) 68° C., 30 sec (annealing step)
(4) 72° C., 30 sec (extension step)
(5) 72° C., 2 min

The steps (2) to (4) were repeated in 40 cycles.

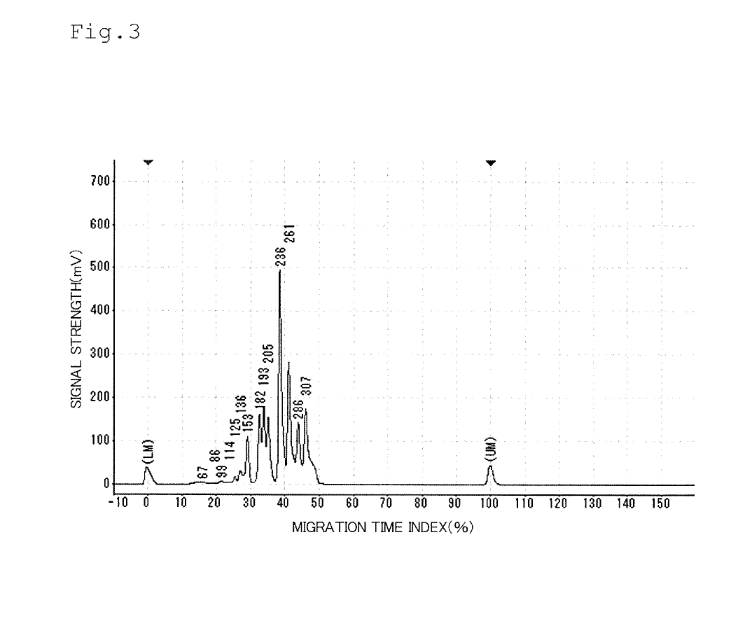

The PCR reaction mixture was analyzed using a microchip electrophoretic device “MultiNA” (manufactured by Shimadzu Corporation) to determine the size of the PCR amplified products. The results are illustrated in FIG. 3.

As illustrated in FIG. 3, eight peaks were detected at an estimated base length of 153 to 307, and it is considered that the amplification target region of the target gene was amplified. However, the estimated base length of the amplified product calculated from the migration distance differs from the estimated base length of the amplified product of each amplification target region. It is conjectured that separation due to electrophoresis was insufficient since the number of amplified products that were close in base length was large.

Therefore, when implementing electrophoretic identification of the amplified products of the target genes that are respectively amplified using the eight primer sets illustrated in FIG. 2 with respect to Escherichia coli, Salmonella, Staphylococcus aureus, and Vibrio parahaemolyticus, it is considered that it is possible to determine the number of peaks, but it is difficult to identify the peak that corresponds to the amplified product of each target gene in practice.

In Test 2, whether or not the eight regions of Escherichia coli, Salmonella, Staphylococcus aureus, and Vibrio parahaemolyticus can be simultaneously detected using the carrier for detecting food poisoning bacteria according to one or more embodiments of the invention was determined.

### Test 2: Identification of Eight Types of Amplified Products Simultaneously Amplified by PCR Using DNA Chip

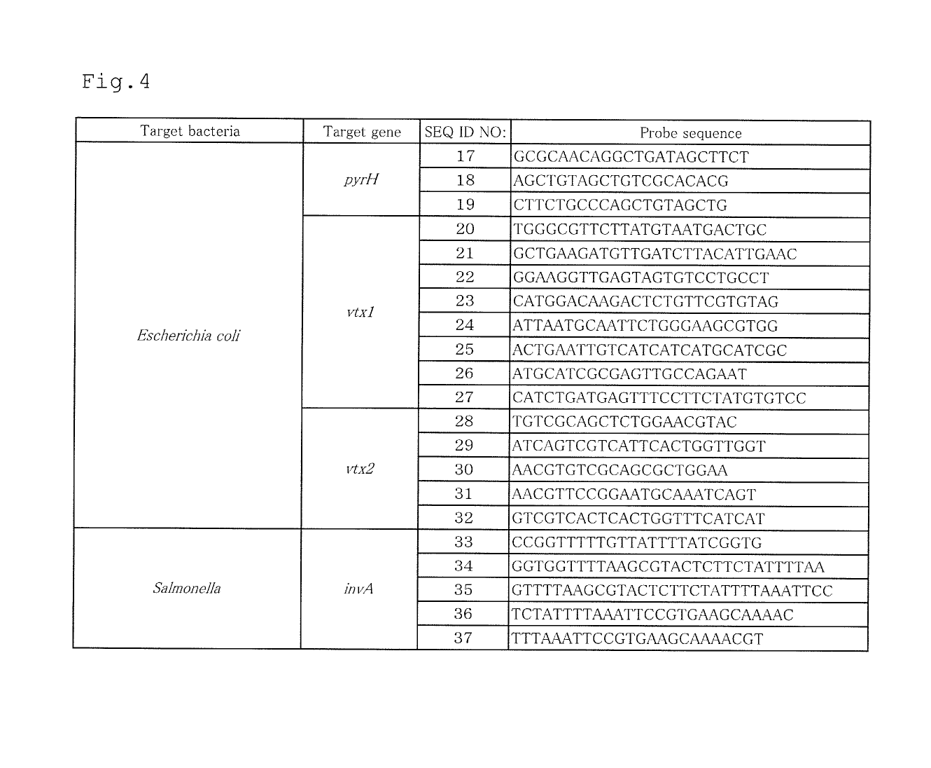

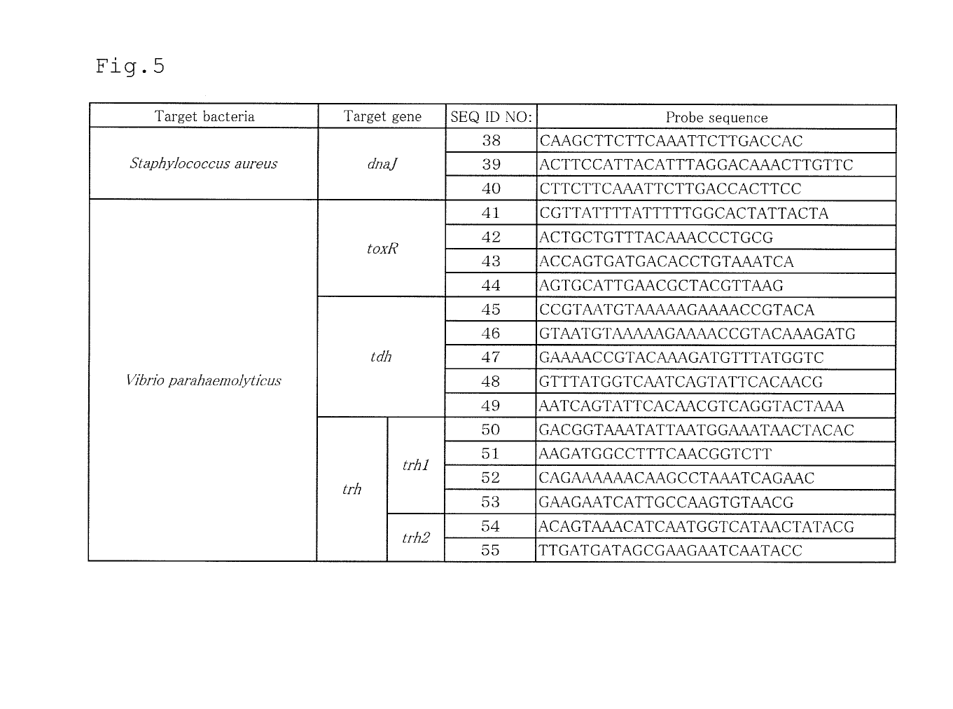

A DNA chip on which probes having the base sequences respectively represented by SEQ ID NO: 17 to 55 illustrated in FIGS. 4 and 5 were immobilized, was prepared.

A mixture prepared by mixing 4 μL of the PCR reaction mixture obtained in Test 1 and 2 μL of a hybridization buffer (3×SSC/0.3% SDS citric acid-physiological saline-sodium dodecyl sulfate), was added dropwise to the DNA chip, and reacted at 45° C. for 1 hour.

After completion of the reaction, the DNA chip was immersed in (washed with) a washing liquid (2×SSC/0.2% SDS solution and 2×SSC solution) at room temperature. After placing cover glass on the DNA chip, fluorescence from the spot region of each probe was detected using a fluorescence detector “BIOSHOT” (manufactured by Toyo Kohan Co., Ltd.).

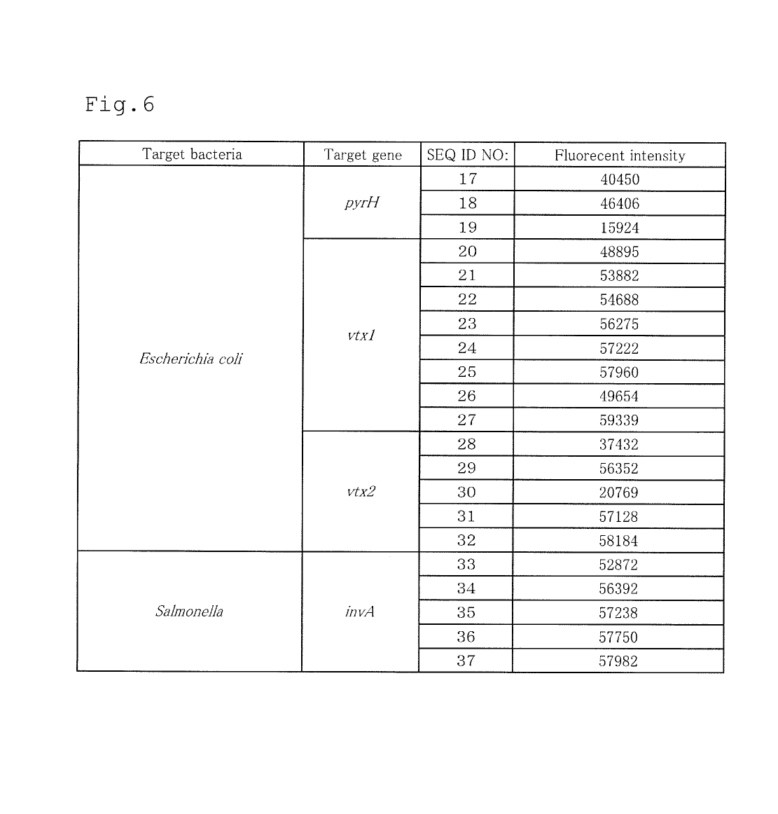

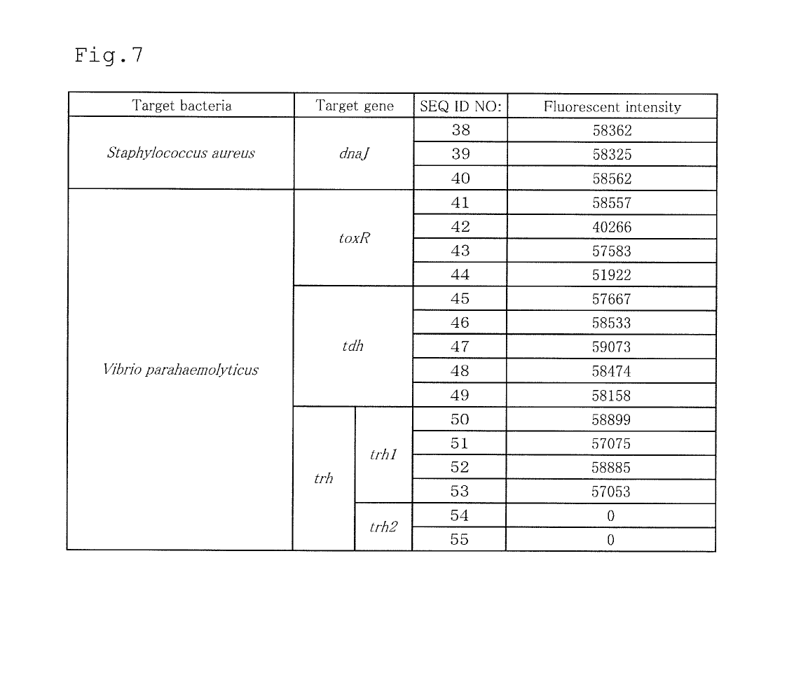

Specifically, the labeling component (Cy5) of the amplified product that hybridized to the probe was caused to emit light by means of laser light excitation, and the light intensity was detected using a CCD camera provided in the detector. The light intensity was converted into an electrical signal, and the electrical signal was converted into a numerical value to obtain fluorescence intensity. The fluorescence intensity (no unit) is an intensity index specific to the device, and was corrected so that the background value was 0. The results are illustrated in FIGS. 6 and 7.

As illustrated in FIGS. 6 and 7, strong fluorescence was detected with respect to the probes respectively selected from the pyrH, vtx1, vtx2, invA, dnaJ, toxR, tdh, and trh1 gene regions. It was thus confirmed that it is possible to simultaneously and specifically detect the amplified products of the eight target genes of Escherichia coli, Salmonella, Staphylococcus aureus, and Vibrio parahaemolyticus using the carrier for detecting food poisoning bacteria according to one or more embodiments of the invention.

### Test 3: Experiment with Regard to Escherichia coli pyrH Probe

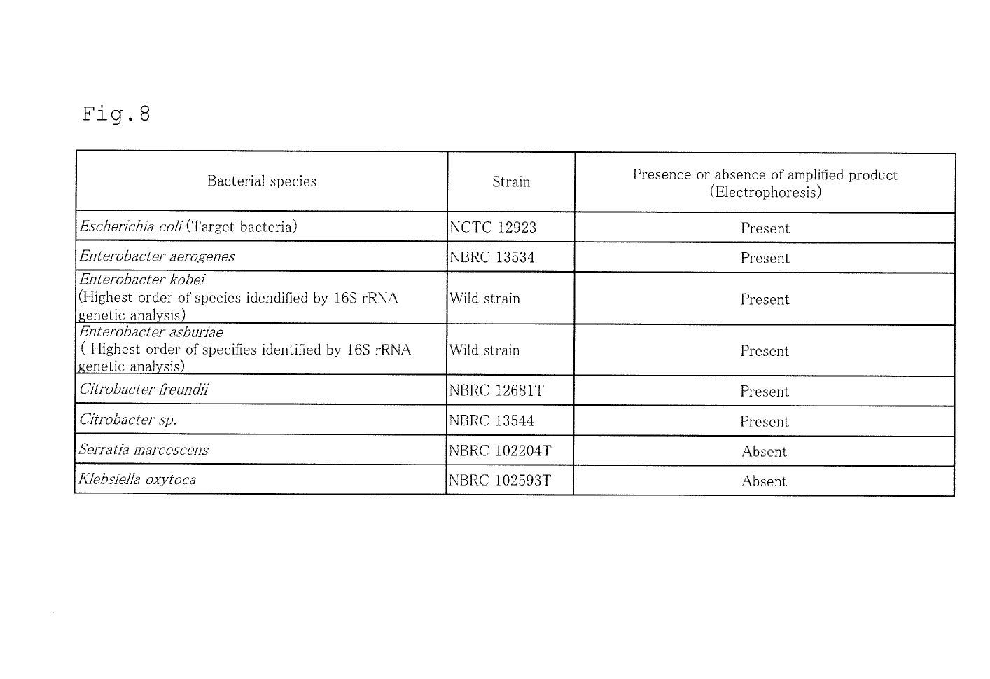

When PCR is performed using the primer set (SEQ ID NO: 1 and 2) that is used to amplify the pyrH region of Escherichia coli, an amplified product based on the genomic DNA of each bacterial species is obtained when bacterial species of Enterobacter and Citrobacter (i.e., species related to Escherichia coli) are included in the sample (see FIG. 8).

In such a case, if the presence or absence of Escherichia coli and enterohemorrhagic Escherichia coli is determined by electrophoresis, a false-positive determination result may be obtained due to the presence of the related species.

In Test 3, whether or not Escherichia coli and the related species can be identified by utilizing the carrier for detecting food poisoning bacteria according to one or more embodiments of the invention was determined. Specifically, the following operation was performed.

The strains of the determination target species illustrated in FIG. 8 were inoculated into a culture medium (“Tryptic Soy Broth” manufactured by Japan Becton, Dickinson and Company), and cultured at 37° C. overnight. 1 mL of the culture was collected, and centrifuged at 5000×g for 10 minutes.

After removing the supernatant liquid, a lysozyme solution (concentration: 20 mg/mL) (20 mM Tris-HCl, pH: 8.0/2 mM EDTA, 1.2% Triton X-100) was added to the precipitate, and the mixture was subjected to a bacteriolysis treatment at 37° C. for 30 minutes. The resulting mixture was subjected to column purification using a DNeasy Blood & Tissue Kit (manufactured by Qiagen) to obtain a DNA extract. The DNA extract was used as a sample subjected to PCR.

In Test 3, a PCR reaction mixture was prepared to have the composition shown below. PCR was performed in the same manner as in Test 1 with respect to each strain to obtain a PCR reaction mixture including the PCR amplified products.
Buffer (10×Ex Taq buffer): 2.0 μL
Nucleic acid substrate (dNTP Mixture): 1.6 μL
Escherichia coli pyrH amplification F primer (Cy5-modified (5′-terminal)): 0.2 μL
Escherichia coli pyrH amplification R primer: 0.2 μL
TaKaRa Ex Taq Hot Start Version: 0.2 μL
DNA in sample: 1.0 μL
Sterilized water: 14.8 μL
(Total amount: 20 μL)

A DNA chip on which probes having the base sequences respectively represented by SEQ ID NO: 17 to 19 illustrated in FIG. 4 were immobilized, was prepared. A mixture prepared by mixing 4 μL of the PCR reaction mixture and 2 μL of a hybridization buffer (3×SSC/0.3% SDS citric acid-physiological saline-sodium dodecyl sulfate), was added dropwise to the DNA chip (on a strain basis), and reacted at 45° C. for 1 hour.

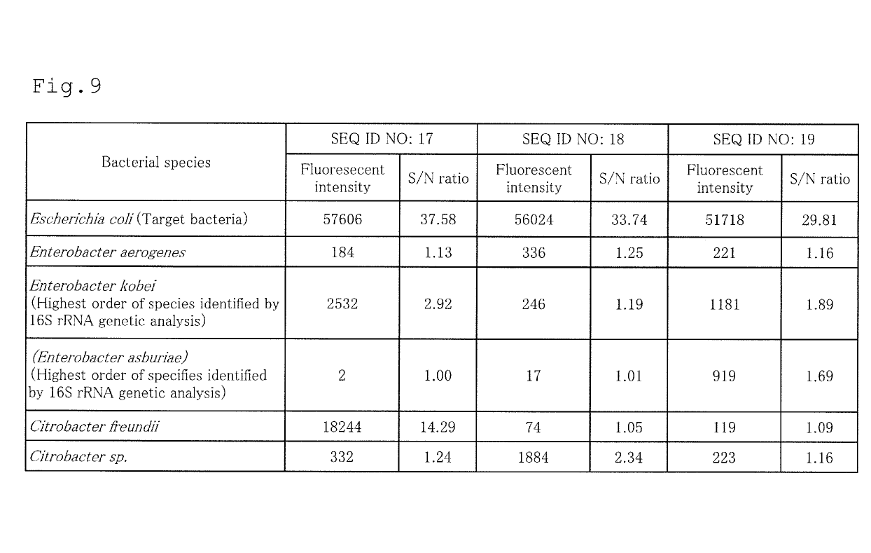

After completion of the reaction, the DNA chip was treated in the same manner as in Test 2 to measure the fluorescence intensity in the spot region of each probe. The S/N ratio was also calculated. The results are illustrated in FIG. 9.

As illustrated in FIG. 9, the probe having the base sequence represented by SEQ ID NO: 17 showed high fluorescence intensity with respect to Escherichia coli (detection target bacteria), and showed relatively high fluorescence intensity with respect to Enterobacter kobei and Citrobacter freundii (non-detection target bacteria). In particular, the S/N ratio with respect to Citrobacter freundii was 3 or more (i.e., a false-positive reaction occurred).

The probe having the base sequence represented by SEQ ID NO: 18 showed high fluorescence intensity with respect to Escherichia coli (detection target bacteria), and showed relatively high fluorescence intensity with respect to Citrobacter sp. (non-detection target bacteria). However, the S/N ratio with respect to Citrobacter sp. was less than 3 (i.e., a false-positive reaction did not occur).

The probe having the base sequence represented by SEQ ID NO: 19 showed high fluorescence intensity with respect to Escherichia coli (detection target bacteria), and showed relatively high fluorescence intensity with respect to Enterobacter kobei (non-detection target bacteria). However, the S/N ratio with respect to Enterobacter kobei was less than 3 (i.e., a false-positive reaction did not occur).

Therefore, the carrier for detecting food poisoning bacteria according to one or more embodiments of the invention may have a configuration in which at least one of the probe having the base sequence represented by SEQ ID NO: 18 and the probe having the base sequence represented by SEQ ID NO: 19 is immobilized on the carrier.

Since the probe having the base sequence represented by SEQ ID NO: 17, the probe having the base sequence represented by SEQ ID NO: 18, and the probe having the base sequence represented by SEQ ID NO: 19 showed high fluorescence intensity with respect to different bacteria, it is possible to reduce or suppress the occurrence of a false-positive determination by combining these probes. Therefore, the carrier for detecting food poisoning bacteria according to one or more embodiments of the invention may have a configuration in which two or more probes among the probe having the base sequence represented by SEQ ID NO: 17, the probe having the base sequence represented by SEQ ID NO: 18, and the probe having the base sequence represented by SEQ ID NO: 19 are immobilized on the carrier, and may have a configuration in which all of the probe having the base sequence represented by SEQ ID NO: 17, the probe having the base sequence represented by SEQ ID NO: 18, and the probe having the base sequence represented by SEQ ID NO: 19 are immobilized on the carrier.

### Test 4: Identification of Thermostable Direct Hemolysin-Related Hemolysin 1 Gene (trh1 Gene) and Thermostable Direct Hemolysin-Related Hemolysin 2 Gene (trh2 Gene)

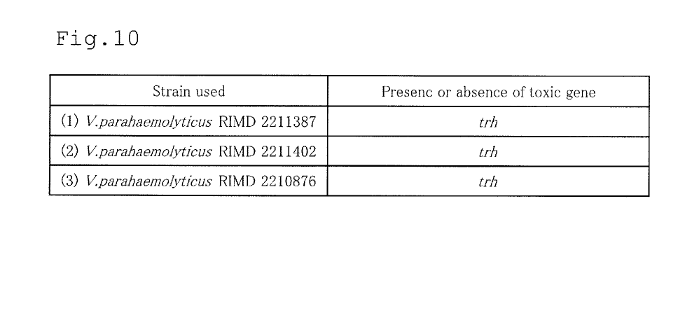

DNA was extracted from the three types of Vibrio parahaemolyticuses strains, for which presence or absence of a toxin gene is known, illustrated in FIG. 10 using an ordinary method. These Vibrio parahaemolyticuses strains were transferred from the Research Institute for Microbial Diseases, Osaka University.

The amplification target regions of the Vibrio parahaemolyticuses strains were simultaneously amplified by multiplex PCR using the trh primer set illustrated in FIG. 2. The resulting amplified products were added dropwise to a DNA chip on which a probe (trh1 probe) for detecting a thermostable direct hemolysin-related hemolysin 1 gene and a probe (trh2 probe) for detecting a thermostable direct hemolysin-related hemolysin 2 gene were immobilized, and whether or not these genes can be identified (detected) was determined. Specifically, the following operation was performed.

The three types of Vibrio parahaemolyticuses strains were inoculated into a culture medium (“Tryptic Soy Broth” manufactured by Japan Becton, Dickinson and Company) (to which 1% sodium chloride was added), and cultured at 37° C. overnight. 1 mL of each culture was collected, mixed, and centrifuged at 5000×g for 10 minutes.

After removing the supernatant liquid, a lysozyme solution (concentration: 20 mg/mL) (20 mM Tris-HCl, pH: 8.0/2 mM EDTA, 1.2% Triton X-100) was added to the precipitate, and a bacteriolysis treatment was performed at 37° C. for 30 minutes. The resulting mixture was subjected to column purification using a DNeasy Blood & Tissue Kit (manufactured by Qiagen) to obtain a DNA extract. The DNA extract was used as a sample subjected to PCR.

The trh gene region was amplified by PCR (on a strain basis) using 10 ng/μL of the DNA extract (strains (1), (2), and (3)) and the trh primer set. A PCR reaction mixture was prepared to have the following composition. Each primer was synthesized by Sigma-Aldrich Co. LLC. on commission, and the other reagents were products manufactured by Takara Bio Inc.
Buffer (10×Ex Taq buffer): 2.0 μL
Nucleic acid substrate (dNTP Mixture): 1.6 μL
Vibrio trh amplification F primer: 0.2 μL
Vibrio trh amplification R primer (Cy5-modified (5′-terminal)): 0.2 μL
TaKaRa Ex Taq Hot Start Version: 0.2 μL
DNA in sample: 1.0 μL
Sterilized water: 14.8 μL
(Total amount: 20 μL)

A DNA chip on which probes (trh1 probes) for detecting a thermostable direct hemolysin-related hemolysin 1 gene having the base sequences respectively represented by SEQ ID NO: 50 to 53, and probes (trh2 probes) for detecting a thermostable direct hemolysin-related hemolysin 2 gene having the base sequences respectively represented by SEQ ID NO: 54 to 55 (see FIG. 5), were immobilized, was prepared. A mixture prepared by mixing 4 μL of the PCR reaction mixture and 2 μL of a hybridization buffer (3×SSC/0.3% SDS citric acid-physiological saline-sodium dodecyl sulfate), was added dropwise to the DNA chip (on a strain basis), and reacted at 45° C. for 1 hour.

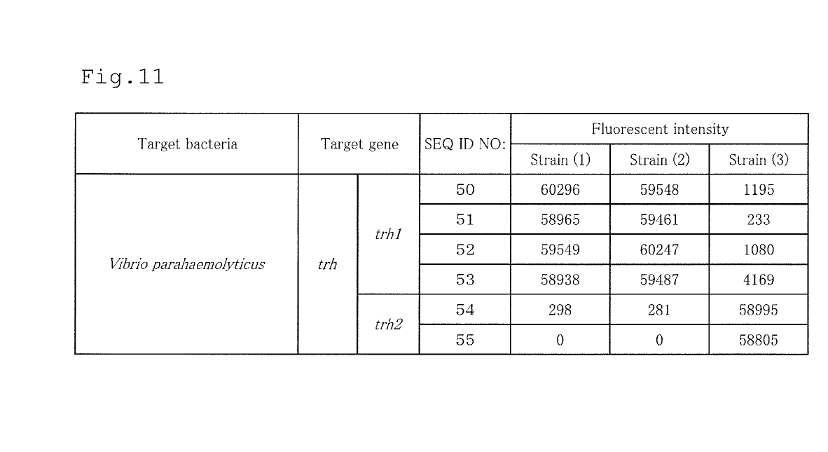

After completion of the reaction, the DNA chip was treated in the same manner as in Test 2 to measure the fluorescence intensity in the spot region of each probe. The results are illustrated in FIG. 11.

As illustrated in FIG. 11, a high fluorescence intensity was obtained by the trh1 detection probes, and only a low fluorescence intensity was obtained by the trh2 detection probes with respect to the strains (1) and (2). It was thus determined that the strains (1) and (2) were pathogenic Vibrio parahaemolyticus including trh1 gene.

A high fluorescence intensity was obtained by the trh2 detection probes, and only a relatively low fluorescence intensity was obtained by the trh1 detection probes with respect to the strain (3). It was thus determined that the strain (3) was pathogenic Vibrio parahaemolyticus including trh2 gene.

It was thus confirmed that it is possible to identify bacteria including a trh 1 gene and bacteria including a trh 2 gene, and avoid a false-negative determination with regard to a trh gene by utilizing the carrier for detecting food poisoning bacteria according to one or more embodiments of the invention on which a trh1 detection probe and a trh2 detection probe are immobilized in combination, when bacteria including the trh gene of pathogenic Vibrio parahaemolyticus are included in the detection target sample.

### Test 5: Determination (1) of Optimum Primer Concentration for Simultaneously Amplifying Amplification Target Regions of Escherichia coli, Salmonella, Staphylococcus aureus, and Vibrio parahaemolyticus

In order to determine an optimum primer concentration for simultaneously amplifying the amplification target regions of Escherichia coli, Salmonella, Staphylococcus aureus, and Vibrio parahaemolyticus, PCR was performed in a state in which the concentration of each primer included in the dnaJ primer set for Staphylococcus aureus in the PCR reaction mixture was set to 150 nM, and the concentration of each primer included in the primer sets for Escherichia coli, Salmonella, and Vibrio parahaemolyticus in the PCR reaction mixture was set to 50, 75, 100, or 150 nM. The details are described below.

A culture medium used for the method for detecting food poisoning bacteria according to one or more embodiments of the invention was prepared as described below. Specifically, a peptone, a yeast extract, magnesium sulfate, sodium chloride, disodium hydrogen phosphate, and potassium dihydrogen phosphate were mixed in the amounts listed below. The mixture was dissolved in distilled water to adjust the pH of the mixture to 7.0. The resulting culture medium was sterilized at 121° C. for 15 minutes using an autoclave.
(Amount per L)
Peptone: 30 g
Yeast extract: 5 g
Magnesium sulfate heptahydrate: 0.5 g
Sodium chloride: 15 g
Disodium hydrogen phosphate: 3.5 g
Potassium dihydrogen phosphate: 1.5 g

After the addition of 25 g of chopped food to 225 mL of the culture medium, Escherichia coli, Salmonella, Staphylococcus aureus, and Vibrio parahaemolyticus strains were inoculated into the culture medium respectively in an amount of about 10 to 50 cfu, and mixed for 30 seconds using a stomachere. The mixture was cultured at 37° C. for 20 hours. A gyoza (i.e., a dumpling with minced pork and vegetable stuffing), precut vegetables, uncured ham, and fish meat sausage were used as the food, and the strains listed below were used as Escherichia coli, Salmonella, Staphylococcus aureus, and Vibrio parahaemolyticus.
Escherichia coli: NCTC 1292
Salmonella: Salmonella enterica subsp. Enterica serovar Abony ACM 5080
Staphylococcus aureus: NCTC 10788
Vibrio parahaemolyticus: RIMD 2210050

These food poisoning bacteria strains were transferred from the following organizations.
NCTC: National Collection of Type Cultures (U.K.)
ACM: Australian Collection of Microorganisms (Australia)
RIMD: Research Institute for Microbial Diseases, Osaka University (Japan)

1 mL of the culture was collected, and centrifuged at 5000×g for 10 minutes. After removing the supernatant liquid, a lysozyme solution (concentration: 20 mg/mL) (20 mM Tris-HCl, pH: 8.0/2 mM EDTA, 1.2% Triton X-100) was added to the precipitate, and the mixture was subjected to a bacteriolysis treatment at 37° C. for 30 minutes. The resulting mixture was subjected to column purification using a DNeasy Blood & Tissue Kit (manufactured by Qiagen) to obtain a DNA extract. The DNA extract was used as a sample subjected to PCR.

The amplification target region of each type of food poisoning bacteria was simultaneously grown by multiplex PCR using four types of primer sets illustrated in FIG. 2 for specifically amplifying the amplification target region of the genomic DNA of each type of food poisoning bacteria, and whether or not the resulting amplified product can be detected was determined. Specifically, the following operation was performed.

A PCR reaction mixture having the following composition was prepared. Each primer was synthesized by Sigma-Aldrich Co. LLC. on commission, and the other reagents were products manufactured by Takara Bio Inc. Multiplex PCR was performed in a state in which the concentration of each primer included in the dnaJ primer set for Staphylococcus aureus was set to 150 nM, and the concentration of each primer included in the primer sets for Escherichia coli, Salmonella, and Vibrio parahaemolyticus was set to 50, 75, 100, or 150 nM.
Buffer (10×Ex Taq buffer): 2.0 μL
Nucleic acid substrate (dNTP Mixture): 1.6 μL
Escherichia coli pyrH F primer (Cy5-modified (5′-terminal)): 0.1, 0.15, 0.2, or 0.3 μL
Escherichia coli pyrH R primer: 0.1, 0.15, 0.2, or 0.3 μL
Salmonella invA F primer: 0.1, 0.15, 0.2, or 0.3 μL
Salmonella invA R primer (Cy5-modified (5′-terminal)): 0.1, 0.15, 0.2, or 0.3 μL
Staphylococcus aureus dnaJ F primer: 0.3 μL
Staphylococcus aureus dnaJ R primer (Cy5-modified (5′-terminal)): 0.3 μL
Vibrio toxR F primer: 0.1, 0.15, 0.2, or 0.3 μL
Vibrio toxR R primer (Cy5-modified (5′-terminal)): 0.1, 0.15, 0.2, or 0.3 μL
TaKaRa Ex Taq Hot Start Version: 0.2 μL
DNA in sample: 1.0 μL
Sterilized water: balance
(Total amount: 20 μL)

PCR gene amplification was performed using a system “Thermal Cycler ep gradient” (manufactured by Eppendorf). The reaction conditions are listed below.
(1) 95° C., 2 min
(2) 95° C., 10 sec (DNA strand separation step (denaturation step))
(3) 68° C., 30 sec (annealing step)
(4) 72° C., 30 sec (DNA synthesis step)
(5) 72° C., 2 min

The steps (2) to (4) were repeated in 40 cycles.

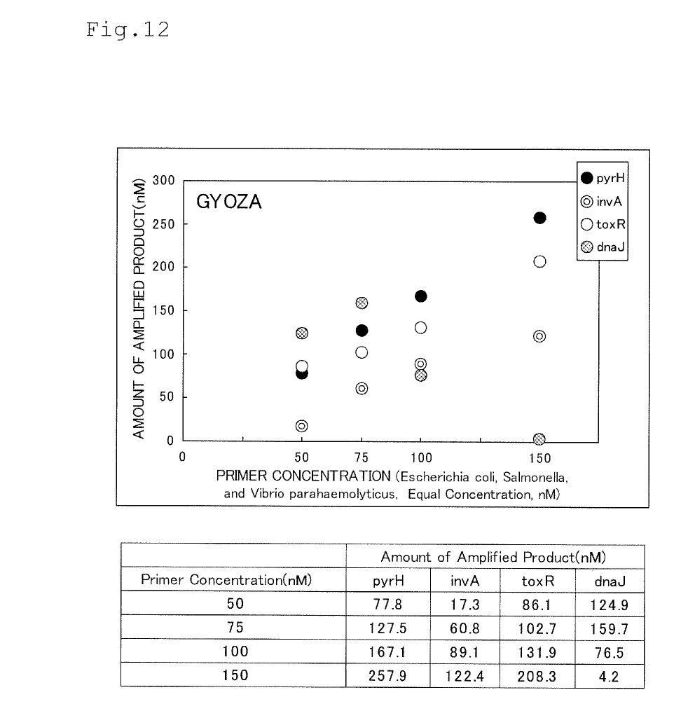

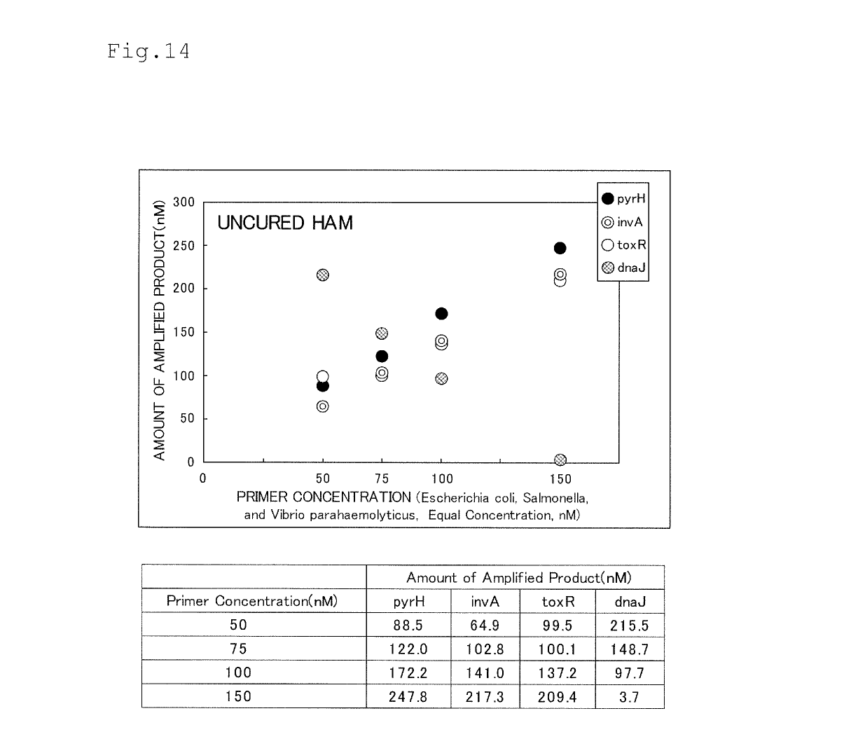

The PCR reaction mixture was subjected to multiplex PCR using a microchip electrophoretic device “MultiNA” (manufactured by Shimadzu Corporation), and the amount of each amplified product was measured. The results are illustrated in FIGS. 12 to 15.

When the concentration of each primer included in the primer sets for Escherichia coli, Salmonella, and Vibrio parahaemolyticus was increased, the amount of the amplified product of the corresponding amplification target region (pyrH, invA, toxR) increased. On the other hand, the amount of the amplified product of the Staphylococcus aureus dnaJ region decreased. It is considered that the amplification efficiency with respect to the pyrH, invA, and toxR regions increased as a result of increasing the concentration of each primer included in the primer sets for Escherichia coli, Salmonella, and Vibrio parahaemolyticus, and the amplification of the dnaJ region was inhibited in a competitive manner.

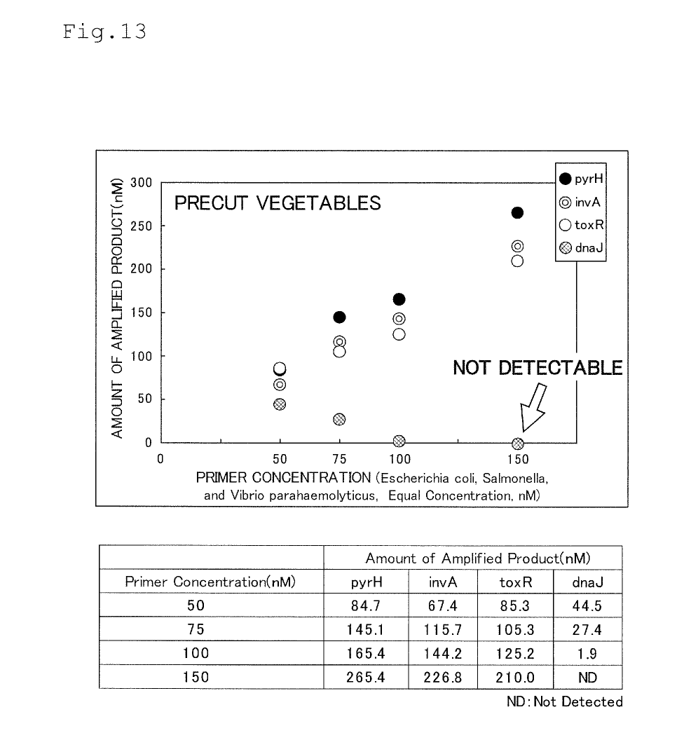

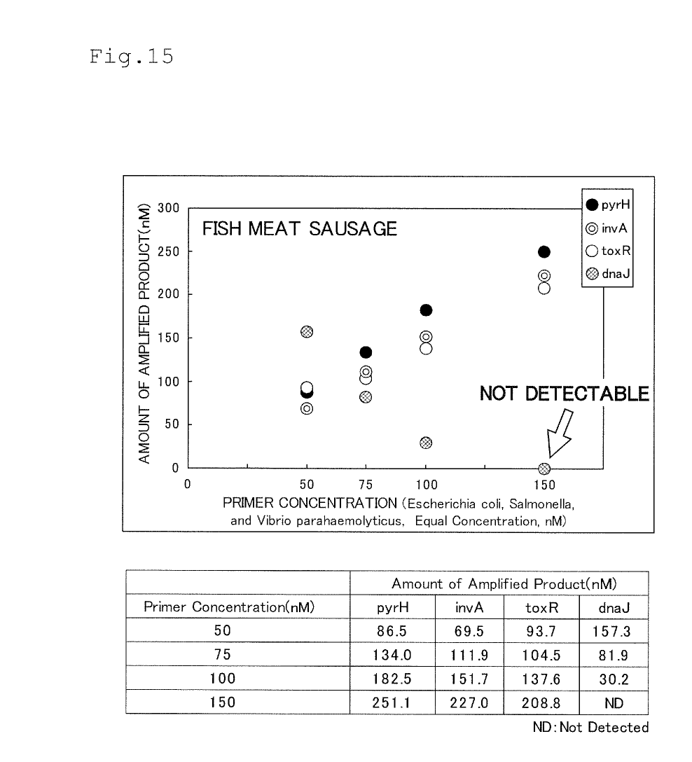

When the concentration of each primer included in the primer sets for Escherichia coli, Salmonella, and Vibrio parahaemolyticus was 150 nM, the amplified product of the dnaJ region could not be detected with respect to precut vegetables (FIG. 13) and fish meat sausage (FIG. 15).

It was thus confirmed that, when simultaneously culturing (growing) Escherichia coli, Salmonella, Staphylococcus aureus, and Vibrio parahaemolyticus using a single culture medium, amplifying each amplification target region by PCR, and detecting each amplified product, the concentration of each primer included in the primer sets for Escherichia coli, Salmonella, and Vibrio parahaemolyticus may be set to 100 nM or less, and for example 50 to 100 nM, in order to simultaneously amplify the amplification target regions (including the dnaJ region) of Escherichia coli, Salmonella, Staphylococcus aureus, and Vibrio parahaemolyticus in a stable manner.

### Test 6: Determination (2) of Optimum Primer Concentration for Simultaneously Amplifying Amplification Target regions of Escherichia coli, Salmonella, Staphylococcus aureus, and Vibrio parahaemolyticus

In order to determine an optimum primer concentration for simultaneously amplifying the amplification target regions of Escherichia coli, Salmonella, Staphylococcus aureus, and Vibrio parahaemolyticus, tests were performed in the same manner as in Test 5, except that the concentration of each primer included in the primer sets for Escherichia coli, Salmonella, and Vibrio parahaemolyticus in the PCR reaction mixture was set to 100 nM, and the concentration of each primer included in the primer set for Staphylococcus aureus in the PCR reaction mixture was set to 50, 75, 100, 125, or 150 nM. The composition of the PCR reaction mixture is shown below.
Buffer (10×Ex Taq buffer): 2.0 μL
Nucleic acid substrate (dNTP Mixture): 1.6 μL
Escherichia coli pyrH F primer (Cy5-modified (5′-terminal)): 0.2 μL
Escherichia coli pyrH R primer: 0.2 μL
Salmonella invA F primer: 0.2 μL
Salmonella invA R primer (Cy5-modified (5′-terminal)): 0.2 μL
Staphylococcus aureus dnaJ F primer: 0.1, 0.15, 0.2, 0.25, or 0.3 μL
Staphylococcus aureus dnaJ R primer (Cy5-modified (5′-terminal)): 0.1, 0.15, 0.2, 0.25, or 0.3 μL
Vibrio toxR F primer: 0.2 μL
Vibrio toxR R primer (Cy5-modified (5′-terminal)): 0.2 μL
TaKaRa Ex Taq Hot Start Version: 0.2 μL
DNA in sample: 1.0 μL
Sterilized water: balance
(Total amount: 20 μL)

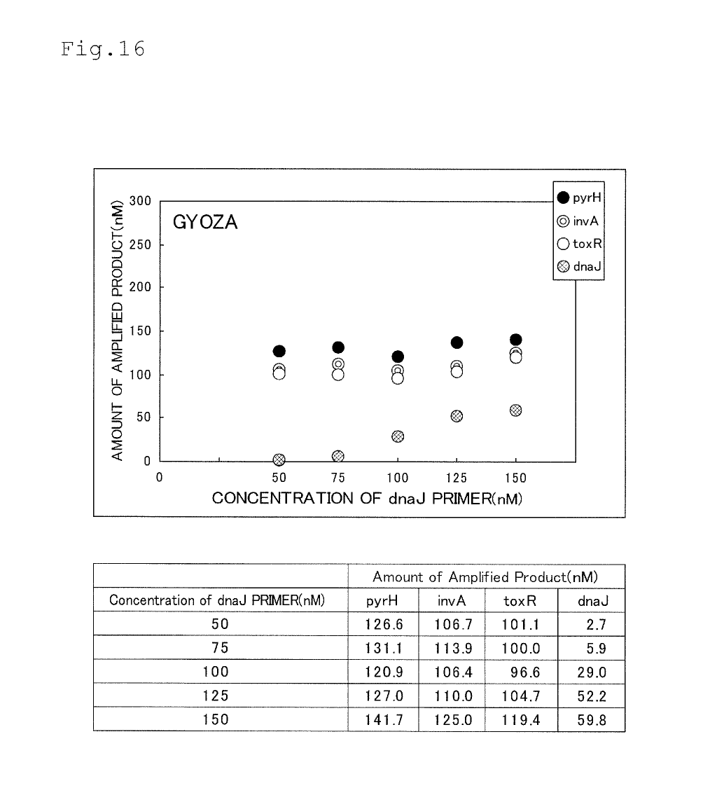

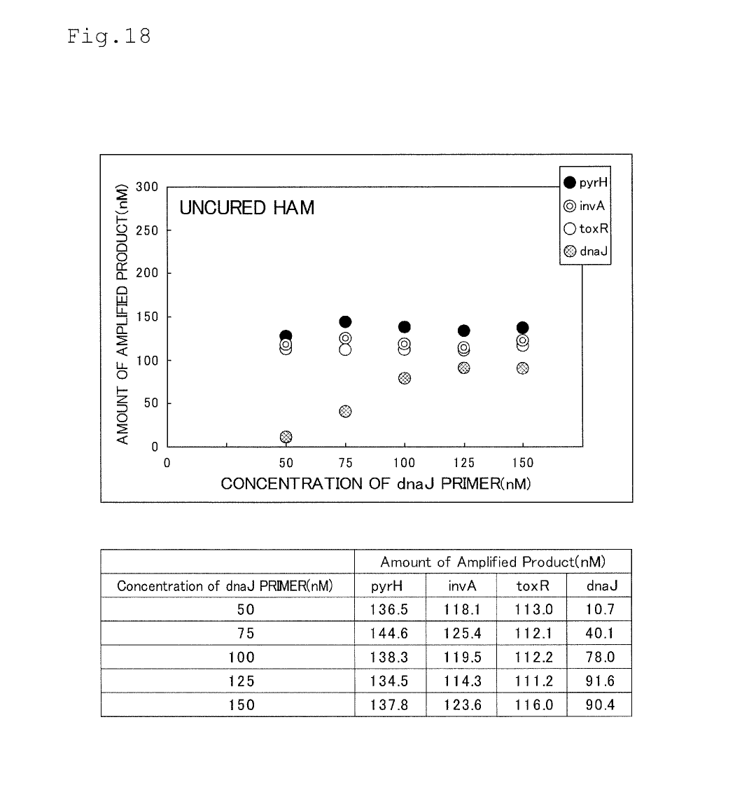

The PCR reaction mixture was subjected to multiplex PCR using a microchip electrophoretic device “MultiNA” (manufactured by Shimadzu Corporation), and the amount of each amplified product was measured. The results are illustrated in FIGS. 16 to 19.

The amount of the amplified product of the dnaJ region tended to increase as the concentration of each primer included in the dnaJ primer for Staphylococcus aureus was increased. On the other hand, the amount of the amplified products of the pyrH, invA, and toxR regions of Escherichia coli, Salmonella, and Vibrio parahaemolyticus were almost constant irrespective of the concentration of each primer included in the dnaJ primer set. Therefore, it is considered that the amplification of the pyrH, invA, and toxR regions was not affected by the amplification of the dnaJ region.

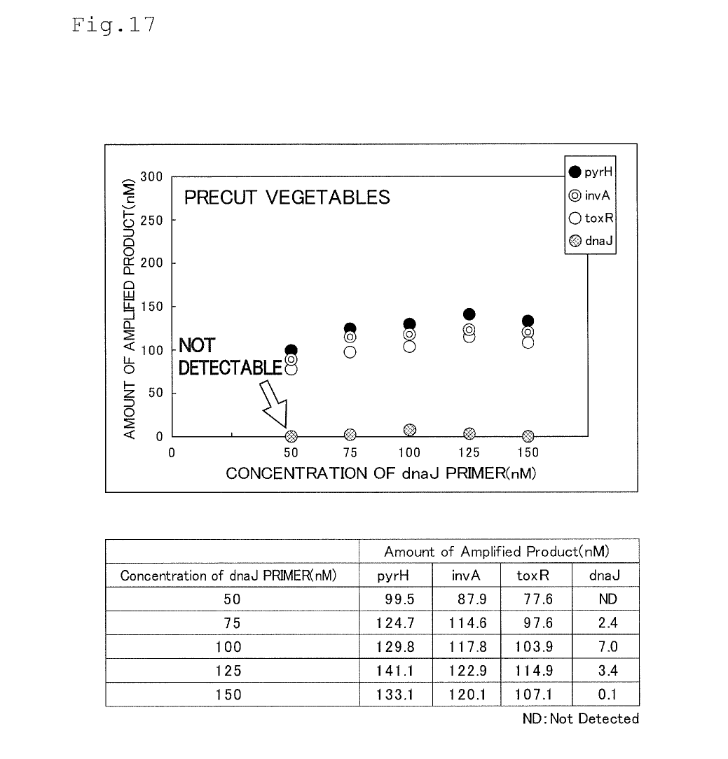

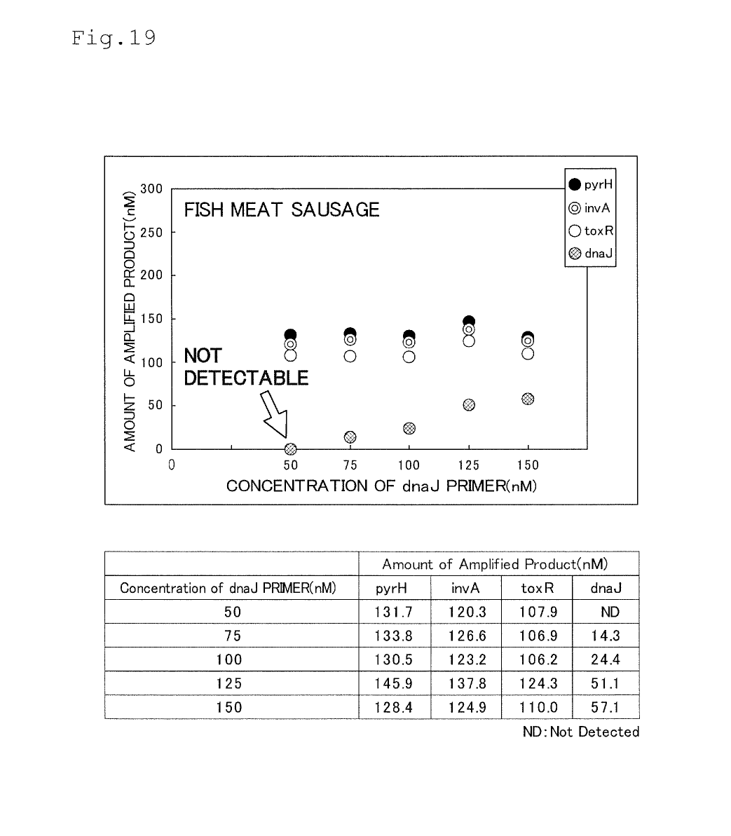

When the concentration of each primer included in the dnaJ primer set for Staphylococcus aureus was 50 nM, the amplified product of the dnaJ region could not be detected with respect to precut vegetables (FIG. 17) and fish meat sausage (FIG. 19). The amount of amplified product was generally small with respect to precut vegetables. On the other hand, a relatively large amount of amplified product was obtained with respect to the other food samples when the primer concentration was 125 nM or more.

It was thus confirmed that, when simultaneously culturing (growing) Escherichia coli, Salmonella, Staphylococcus aureus, and Vibrio parahaemolyticus using a single culture medium, amplifying each amplification target region by PCR, and detecting each amplified product, the concentration of each primer included in the dnaJ primer set for detecting Staphylococcus aureus may be set to 75 nM or more (for example, 125 nM or more, and 150 nM or more) in order to simultaneously amplify the amplification target regions (including the dnaJ region) of Escherichia coli, Salmonella, Staphylococcus aureus, and Vibrio parahaemolyticus in a stable manner.

The invention is not limited to the embodiments and the examples described above. Various modifications may be made of the embodiments and the examples described above without departing from the scope of the invention. For example, an additional probe (other than those described above) may be further immobilized on the carrier for detecting food poisoning bacteria according to one or more embodiments of the invention, or an additional component may be added to the PCR reaction mixture used for the kit for detecting food poisoning bacteria according to one or more embodiments of the invention.

### INDUSTRIAL APPLICABILITY

One or more embodiments of the invention may suitably be used to simultaneously and specifically detect Escherichia coli, Salmonella, Staphylococcus aureus, and Vibrio parahaemolyticus in the fields of food inspection, epidemiological environmental inspection, environmental inspection, clinical examination, livestock health management, and the like.

1. A carrier for detecting food poisoning bacteria that is used to simultaneously detect Escherichia coli, Salmonella, Staphylococcus aureus, and Vibrio parahaemolyticus, the carrier comprising a plurality of probes comprising:
a probe from a region of a uridine monophosphate kinase gene (pyrH gene) of Escherichia coli;
a probe from a region of a verotoxin 1 gene (vtx1 gene) of Escherichia coli;
a probe from a region of a verotoxin 2 gene (vtx2 gene) of Escherichia coli;
a probe from a region of an invasive factor-related gene (invA gene) of Salmonella;
a probe from a region of a heat shock protein gene (dnaJ gene) of Staphylococcus aureus;
a probe from a region of a virulence regulatory gene (toxR gene) of Vibrio parahaemolyticus;
a probe from a region of a thermostable direct hemolysin gene (tdh gene) of Vibrio parahaemolyticus;
a probe from a region of a thermostable direct hemolysin-related hemolysin 1 gene (trh1 gene) of Vibrio parahaemolyticus; and
a probe from a region of a thermostable direct hemolysin-related hemolysin 2 gene (trh2 gene) of Vibrio parahaemolyticus, wherein the plurality of probes are immobilized on the carrier.

2. The carrier for detecting food poisoning bacteria according to claim 1,
wherein the probe from the region of the pyrH gene comprises a base sequence selected from the group consisting of SEQ ID NO: 17 to 19,
the probe from the region of the vtx1 gene comprises a base sequence selected from the group consisting of SEQ ID NO: 20 to 27,
the probe from the region of the vtx2 gene comprises a base sequence selected from the group consisting of SEQ ID NO: 28 to 32,
the probe from the region of the invA gene comprises a base sequence selected from the group consisting of SEQ ID NO: 33 to 37,
the probe from the region of the dnaJ gene comprises a base sequence selected from the group consisting of SEQ ID NO: 38 to 40,
the probe from the region of the toxR gene comprises a base sequence selected from the group consisting of SEQ ID NO: 41 to 44,
the probe from the region of the tdh gene comprises a base sequence selected from the group consisting of SEQ ID NO: 45 to 49,
the probe from the region of the trh1 gene comprises a base sequence selected from the group consisting of SEQ ID NO: 50 to 53, and
the probe from the region of the trh2 gene comprises a base sequence selected from the group consisting of SEQ ID NO: 54 and 55.

3. The carrier for detecting food poisoning bacteria according to claim 2, wherein at least one of the plurality of probes is
(1) a probe having the corresponding base sequence, wherein one base or a plurality of bases are deleted, substituted, or inserted,
(2) a probe that hybridizes to a nucleic acid fragment having a base sequence complementary to the corresponding base sequence under stringent conditions, or
(3) a probe having a base sequence complementary to that of the probe as defined in (1) or (2).

4. A kit for detecting food poisoning bacteria that is used to simultaneously detect Escherichia coli, Salmonella, Staphylococcus aureus, and Vibrio parahaemolyticus, the kit comprising:
the carrier for detecting food poisoning bacteria according to claim 1; and a PCR reaction mixture, wherein the PCR reaction mixture comprises:
a pyrH primer set that is used to amplify a DNA fragment comprising the region of the uridine monophosphate kinase gene (pyrH gene) of Escherichia coli, wherein the pyrH primer set comprises a primer having the base sequence represented by SEQ ID NO: 1 and a primer having the base sequence represented by SEQ ID NO: 2;
a vtx1 primer set comprising a primer having the base sequence represented by SEQ ID NO: 3 and a primer having the base sequence represented by SEQ ID NO: 4;
a vtx2 primer set comprising a primer having the base sequence represented by SEQ ID NO: 5 and a primer having the base sequence represented by SEQ ID NO: 6;
an invA primer set comprising a primer having the base sequence represented by SEQ ID NO: 7 and a primer having the base sequence represented by SEQ ID NO: 8;
a dnaJ primer set comprising a primer having the base sequence represented by SEQ ID NO: 9 and a primer having the base sequence represented by SEQ ID NO: 10;
a toxR primer set comprising a primer having the base sequence represented by SEQ ID NO: 11 and a primer having the base sequence represented by SEQ ID NO: 12;
a tdh primer set comprising a primer having the base sequence represented by SEQ ID NO: 13 and a primer having the base sequence represented by SEQ ID NO: 14; and
a trh primer set comprising a primer having the base sequence represented by SEQ ID NO: 15 and a primer having the base sequence represented by SEQ ID NO: 16, and
wherein a primer concentration of the dnaJ primer set in the PCR reaction mixture is higher than a primer concentration of pyrH primer set, a primer concentration of the vtx1 primer set, a primer concentration of the vtx2 primer set, a primer concentration of the invA primer set, a primer concentration of the toxR primer set, a primer concentration of the tdh primer set, and a primer concentration of the trh primer set in the PCR reaction mixture by a factor of 1.25 or more.

5. The kit for detecting food poisoning bacteria according to claim 4, wherein the primer concentration of the dnaJ primer set is 125 nM or more, and the primer concentration of the pyrH primer set, the primer concentration of the vtx1 primer set, the primer concentration of the vtx2 primer set, the primer concentration of the invA primer set, the primer concentration of the toxR primer set, the primer concentration of the tdh primer set, and the primer concentration of the trh primer set are 50 to 100 nM.

6. A method for detecting food poisoning bacteria that simultaneously detects Escherichia coli, Salmonella, Staphylococcus aureus, and Vibrio parahaemolyticus, the method comprising:
simultaneously enriching Escherichia coli, Salmonella, Staphylococcus aureus, and Vibrio parahaemolyticus in a culture medium;
extracting genomic DNA of Escherichia coli, Salmonella, Staphylococcus aureus, and Vibrio parahaemolyticus from the culture medium subjected to the enrichment;
performing PCR for simultaneous amplification of a DNA fragment comprising a region of a uridine monophosphate kinase gene (pyrH gene) of Escherichia coli, a DNA fragment comprising a region of an invasive factor-related gene (invA gene) of Salmonella, a DNA fragment comprising a region of a heat shock protein gene (dnaJ gene) of Staphylococcus aureus, and a DNA fragment comprising a region of virulence regulatory gene (toxR) of Vibrio parahaemolyticus; and
simultaneously detecting amplified products obtained by the amplification by electrophoresis or a DNA chip,
wherein a primer concentration of a dnaJ primer set that is used to amplify the DNA fragment of the dnaJ gene in a PCR reaction mixture used in the amplification is higher than a primer concentration of a pyrH primer set that is used to amplify the DNA fragment of the pyrH gene, a primer concentration of an invA primer set that is used to amplify the DNA fragment of the invA gene, and a primer concentration of a toxR primer set that is used to amplify the DNA fragment of the toxR gene in the PCR reaction mixture by a factor of 1.25 or more.

7. The method for detecting food poisoning bacteria according to claim 6, wherein the primer concentration of the dnaJ primer set is 125 nM or more, and the primer concentration of the pyrH primer set, the primer concentration of the invA primer set, and the primer concentration of the toxR primer set are 50 to 100 nM.

8. The method for detecting food poisoning bacteria according to claim 6, wherein the culture medium comprises a peptone, a yeast extract, magnesium sulfate, and sodium chloride.

9. The method for detecting food poisoning bacteria according to claim 6, wherein
the pyrH primer set comprises a primer having the base sequence represented by SEQ ID NO: 1, and a primer having the base sequence represented by SEQ ID NO: 2,
the invA primer set comprises a primer having the base sequence represented by SEQ ID NO: 7, and a primer having the base sequence represented by SEQ ID NO: 8,
the dnaJ primer set comprises a primer having the base sequence represented by SEQ ID NO: 9 and a primer having the base sequence represented by SEQ ID NO: 10, and
the toxR primer set comprises a primer having the base sequence represented by SEQ ID NO: 11 and a primer having the base sequence represented by SEQ ID NO: 12.

10. A PCR reaction mixture for food poisoning bacteria that is used to simultaneously amplify Escherichia coli, Salmonella, Staphylococcus aureus, and Vibrio parahaemolyticus by PCR, the PCR reaction mixture comprising:
a pyrH primer set comprising a primer having the base sequence represented by SEQ ID NO: 1 and a primer having the base sequence represented by SEQ ID NO: 2;
an invA primer set comprising a primer having the base sequence represented by SEQ ID NO: 7 and a primer having the base sequence represented by SEQ ID NO: 8;
a dnaJ primer set comprising a primer having the base sequence represented by SEQ ID NO: 9 and a primer having the base sequence represented by SEQ ID NO: 10; and
a toxR primer set comprising a primer having the base sequence represented by SEQ ID NO: 11 and a primer having the base sequence represented by SEQ ID NO: 12,
wherein a primer concentration of the dnaJ primer set in the PCR reaction mixture is 125 nM or more, and a primer concentration of the pyrH primer set, a primer concentration of the invA primer set, and a primer concentration of the toxR primer set in the PCR reaction mixture are 50 to 100 nM.


SEQUENCE LISTING (55 sequences)

SEQ ID NO: 1 - DNA, 31 bp, Escherichia coli
cgctcgtctgatgtccgctattccattgaat

SEQ ID NO: 2 - DNA, 26 bp, Escherichia coli
accacgcaggcaagctgctgagtcgg

SEQ ID NO: 3 - DNA, 33 bp, Escherichia coli
tgttactgtgacagctgaagctttacgttttcg

SEQ ID NO: 4 - DNA, 32 bp, Escherichia coli
tcccacaatattttattgtgcgtaatcccacg

SEQ ID NO: 5 - DNA, 27 bp, Escherichia coli
ggtttccatgacaacggacagcagtta

SEQ ID NO: 6 - DNA, 31 bp, Escherichia coli
gtccactgaactccattaacgccagatatga

SEQ ID NO: 7 - DNA, 30 bp, Salmonella sp.
gaacaacccatttgtattggttgttacggc

SEQ ID NO: 8 - DNA, 23 bp, Salmonella sp.
ggctgctcgcctttgctggtttt

SEQ ID NO: 9 - DNA, 24 bp, Staphylococcus aureus
cacgcctggagagccttcaccagc

SEQ ID NO: 10 - DNA, 30 bp, Staphylococcus aureus
gttactgtaatggcgctggtcatgtagctg

SEQ ID NO: 11 - DNA, 25 bp, Vibrio parahaemolyticus
ctgctgtgaatccttggattccacg

SEQ ID NO: 12 - DNA, 27 bp, Vibrio parahaemolyticus
tcacgttctcatacgagtggttgctgt

SEQ ID NO: 13 - DNA, 33 bp, Vibrio parahaemolyticus
cggttctgatgagatattgtttgttgttcgaga

SEQ ID NO: 14 - DNA, 34 bp, Vibrio parahaemolyticus
tcattaatgttcacagtcatgtaggatgtcagcc

SEQ ID NO: 15 - DNA, 31 bp, Vibrio parahaemolyticus
ttcacaactgcaggctcaaaatggttaagcg

SEQ ID NO: 16 - DNA, 31 bp, Vibrio parahaemolyticus
caaacatatgcccatttccgctctcatatgc

SEQ ID NO: 17 - DNA, 22 bp, Escherichia coli
gcgcaacaggctgatagcttct

SEQ ID NO: 18 - DNA, 20 bp, Escherichia coli
agctgtagctgtcgcacacg

SEQ ID NO: 19 - DNA, 20 bp, Escherichia coli
cttctgcccagctgtagctg

SEQ ID NO: 20 - DNA, 24 bp, Escherichia coli
tgggcgttcttatgtaatgactgc

SEQ ID NO: 21 - DNA, 27 bp, Escherichia coli
gctgaagatgttgatcttacattgaac

SEQ ID NO: 22 - DNA, 24 bp, Escherichia coli
ggaaggttgagtagtgtcctgcct

SEQ ID NO: 23 - DNA, 25 bp, Escherichia coli
catggacaagactctgttcgtgtag

SEQ ID NO: 24 - DNA, 25 bp, Escherichia coli
attaatgcaattctgggaagcgtgg

SEQ ID NO: 25 - DNA, 26 bp, Escherichia coli
actgaattgtcatcatcatgcatcgc

SEQ ID NO: 26 - DNA, 22 bp, Escherichia coli
atgcatcgcgagttgccagaat

SEQ ID NO: 27 - DNA, 28 bp, Escherichia coli
catctgatgagtttccttctatgtgtcc

SEQ ID NO: 28 - DNA, 21 bp, Escherichia coli
tgtcgcagctctggaacgtac

SEQ ID NO: 29 - DNA, 24 bp, Escherichia coli
atcagtcgtcattcactggttggt

SEQ ID NO: 30 - DNA, 20 bp, Escherichia coli
aacgtgtcgcagcgctggaa

SEQ ID NO: 31 - DNA, 23 bp, Escherichia coli
aacgttccggaatgcaaatcagt

SEQ ID NO: 32 - DNA, 24 bp, Escherichia coli
gtcgtcactcactggtttcatcat

SEQ ID NO: 33 - DNA, 24 bp, Salmonella sp.
ccggtttttgttattttatcggtg

SEQ ID NO: 34 - DNA, 30 bp, Salmonella sp.
ggtggttttaagcgtactcttctattttaa

SEQ ID NO: 35 - DNA, 31 bp, Salmonella sp.
gttttaagcgtactcttctattttaaattcc

SEQ ID NO: 36 - DNA, 27 bp, Salmonella sp.
tctattttaaattccgtgaagcaaaac

SEQ ID NO: 37 - DNA, 24 bp, Salmonella sp.
tttaaattccgtgaagcaaaacgt

SEQ ID NO: 38 - DNA, 25 bp, Staphylococcus aureus
caagcttcttcaaattcttgaccac

SEQ ID NO: 39 - DNA, 30 bp, Staphylococcus aureus
acttccattacatttaggacaaacttgttc

SEQ ID NO: 40 - DNA, 25 bp, Staphylococcus aureus
cttcttcaaattcttgaccacttcc

SEQ ID NO: 41 - DNA, 28 bp, Vibrio parahaemolyticus
cgttattttatttttggcactattacta

SEQ ID NO: 42 - DNA, 22 bp, Vibrio parahaemolyticus
actgctgtttacaaaccctgcg

SEQ ID NO: 43 - DNA, 24 bp, Vibrio parahaemolyticus
accagtgatgacacctgtaaatca

SEQ ID NO: 44 - DNA, 23 bp, Vibrio parahaemolyticus
agtgcattgaacgctacgttaag

SEQ ID NO: 45 - DNA, 26 bp, Vibrio parahaemolyticus
ccgtaatgtaaaaagaaaaccgtaca

SEQ ID NO: 46 - DNA, 30 bp, Vibrio parahaemolyticus
gtaatgtaaaaagaaaaccgtacaaagatg

SEQ ID NO: 47 - DNA, 27 bp, Vibrio parahaemolyticus
gaaaaccgtacaaagatgtttatggtc

SEQ ID NO: 48 - DNA, 27 bp, Vibrio parahaemolyticus
gtttatggtcaatcagtattcacaacg

SEQ ID NO: 49 - DNA, 29 bp, Vibrio parahaemolyticus
aatcagtattcacaacgtcaggtactaaa

SEQ ID NO: 50 - DNA, 30 bp, Vibrio parahaemolyticus
gacggtaaatattaatggaaataactacac

SEQ ID NO: 51 - DNA, 22 bp, Vibrio parahaemolyticus
aagatggcctttcaacggtctt

SEQ ID NO: 52 - DNA, 26 bp, Vibrio parahaemolyticus
cagaaaaaacaagcctaaatcagaac

SEQ ID NO: 53 - DNA, 24 bp, Vibrio parahaemolyticus
gaagaatcattgccaagtgtaacg

SEQ ID NO: 54 - DNA, 30 bp, Vibrio parahaemolyticus
acagtaaacatcaatggtcataactatacg

SEQ ID NO: 55 - DNA, 25 bp, Vibrio parahaemolyticus
ttgatgatagcgaagaatcaatacc